(19) United States
(12) Patent Application Publication
(10) Pub. No.: US 2009/0092603 A1
(43) Pub. Date: Apr. 9, 2009

(54) EARLY DIAGNOSIS AND TREATMENT OF DRUG RESISTANCE IN MUC1-POSITIVE CANCER

(76) Inventor: Cynthia C. BAMDAD, Boston, MA (US)

Correspondence Address: JHK LAW, P.O. BOX 1078, LA CANADA, CA, 91012-1078, US

(21) Appl. No.: 12/238,321

(22) Filed: Sep. 25, 2008

Related U.S. Application Data
(60) Provisional application No. 60/975,136, filed on Sep. 25, 2007.

(51) Int. Cl. A61K 39/395; C12Q 1/68; A61P 35/00

(52) U.S. Cl. 424/133.1; 435/6

(57) ABSTRACT

A method of determining likelihood of acquiring drug resistance of a tumor or cancerous cells, cancer metastasis, cancer recurrence, or decreased life expectancy, comprising measuring the level of MUC1 or MUC1-associated factor expressed in the cancerous cells or tumor.

### CROSS-REFERENCE TO RELATED APPLICATIONS

The present application claims the benefit of priority to U.S. Provisional Application No. 60/975,136, filed Sep. 25, 2007, the contents of which are incorporated by reference in their entirety.

### BACKGROUND OF THE INVENTION

1. Field of the Invention

The present invention relates to a method of diagnosing cancer. The present invention also relates to a method of determining cells or tumors that have become resistant to treatment with cancer drugs. The present invention also relates to a method of treating cancer by inhibiting the effects of MUC1*.

2. General Background and State of the Art:

Epithelial cancers, which include breast, prostate, colon, and lung cancers are the most common cancers in adults. Over 75% of all solid tumor cancers are characterized by the aberrant expression of the MUC1 receptor (Development and characterization of breast cancer reactive monoclonal antibodies directed to the core protein of the human milk mucin Burchell J, Gendler S, Taylor-Papadimitriou J, Girling A, Lewis A, Millis R, and Lamport D. (1987) Cancer Res., 47, 5476-5482; Monoclonal antibodies to epithelial sialomucins recognize epitopes at different cellular sites in adenolymphomas of the parotid gland Zotter S, Hageman P C, Lossnitzer A, Mooi W and Hilgers J (1988) Cancer Rev. 11-12, 55-101; Mucins and mucin binding proteins in colorectal cancer Byrd J C and Bresalier R S (2004) Cancer Metastasis Review January-June; 23(1-2):77-99), wherein aberrant expression means that the receptor is no longer localized to the apical border of luminal cells but rather is uniformly distributed over the entire cell surface (Differential reactivity of a novel monoclonal antibody (DF3) with human malignant versus benign breast tumors (1984) Kufe D, Inghirami G, Abe, M, Hayes D, Justi-wheeler H and Schlom J Hybridoma, 3, 223-232). The greatest percentage of MUC1-positive cancers is in breast cancers where greater than 96% show aberrant MUC1 expression. Interestingly, in the adult female, breast tissue must undergo cyclic bursts of growth and apoptosis with each menstrual period and pregnancy. Thus it follows that breast tissue must maintain functional stem cell or at least progenitor cell populations throughout adult female life.

In the United States, 211,000 women are diagnosed each year with breast cancer. Of the 42,000 breast cancer patients who overexpress the HER2 growth factor receptor, less than 35% are responsive to treatment with the HER2-disabling antibody, called HERCEPTIN®. Despite those statistics, women diagnosed with breast cancer are now tested to determine how much of this important growth factor receptor is present in their tumor because patients whose treatment includes HERCEPTIN® are three-times more likely to survive and two-times more likely to survive without a cancer recurrence. Because HERCEPTIN® only works for patients whose tumors overexpress HER2, insurance only covers treatment if their biopsy confirms HER2 overexpression. The phenomenon of acquired multi-drug resistance occurs in many cancer patients after treatment with standard chemo therapy and typically signals the end of hope for successful medical intervention. It is striking that 25% of the HER2 metastatic breast cancer patients acquire resistance to HERCEPTIN® within the first year of treatment. The prognosis for these women is bleak and the reason for this early acquisition of HERCEPTIN® resistance has been unclear.

MUC1 (mucin 1) is a transmembrane mucin glycoprotein that is expressed on a number of epithelial cell types (Molecular cloning and expression of the human tumor associated polymorphic epithelial mucin, PEM. Gendler Sj, Lancaster C A, Taylor-Papadimitriou J, Dhuig, T, Peat, N, Burchell, J, Pemberton, L, Lalani, E-N and Wilson D. (1990) J. Biol. Chem. 265, 15286-15293; Episialin, a carcinoma associated mucin, is generated by a polymorphic gene encoding splice variants with alternative amino termini. Ligtenberg M J L, Vos H L, Genissen, A M C and Hilkens J. (1990) J. Biol. Chem. 265, 15573-15578), on haematopoietic cells (Evaluation of MUC1 and EGP40 in Bone marrow and Peripheral Blood as a Marker for Occult breast cancer (2001 Zhong X Y, Kaul S, Bastert G, Arch Gynecol Obstet 264:177-181), and on progenitor cells as well (Epithelial Progenitors in the Normal Human mammary Gland. Stingl J, Raouf A, Emerman J, and Eaves C. (2005). Journal of Mammary Gland Biology and Neoplasia, Vol. 10, No. 1, 49-59). The cell surface receptor MUC1 is present at the apical border of healthy epithelium, but is aberrantly expressed (spread over the entire cell surface) in a wide range of human solid tumors. It has been known for some time that the MUC1 receptor can be “shed” from the cell surface, as a portion of the extracellular domain can be detected in the blood of breast cancer patients. The inventors previously disclosed that the portion of the MUC1 receptor that remains attached to the cell surface after cleavage, consisting primarily of PSMGFR, is the major growth factor receptor that mediates the growth of MUC1-positive cancer cells in vitro. Transfection of a variant MUC1 receptor comprised of the intact transmembrane and cytoplasmic domains, but having an ectodomain that terminates at the end of the PSMGFR sequence is sufficient to confer the ability of these cells to grow anchorage-independently.

In further detail, MUC1 comprises several regions termed herein as follows, recited in an order starting from the C-terminus and extending through the cell membrane and out into the extracellular domain. The basic structure of the MUC1 receptor comprises: 1) cytoplasmic tail; 2) transmembrane section; 3) MGFR; 4) IBR, 5) Unique Region, 6) repeats, and N-terminus region comprising a signal peptide. For a detailed description of MUC1 and its function in normal and tumor cells, see PCT/US2005/032821, which is incorporated by reference herein, in its entirety for its description of the function and activity of cleaved MUC1 on the cell surface.

It would be advantageous to establish a method for identifying which patients are at risk for acquiring early resistance to drugs. It would also be advantageous to develop treatment protocols that would reduce their risk for acquiring early drug resistance and for rescuing patients at the first indication of acquisition of drug resistance.

### SUMMARY OF THE INVENTION

In one aspect, the invention is directed to a method of determining likelihood of acquiring drug resistance of a tumor or cancerous cells, cancer metastasis, cancer recurrence, or decreased life expectancy, comprising measuring the level of MUC1 or MUC1-associated factor expressed in the cancerous cells or tumor. The method may comprise measuring an increase in the amount of MUC1 or MUC1-associated factor that the cancerous cells or tumor produces. Fluorescent in situ hybridization (FISH) technique may be used to measure the ratio of MUC1 expression relative to CEP17 expression, wherein a ratio of greater than 1.8 indicates likelihood of acquiring drug resistance of a tumor or cancerous cells, cancer metastasis, cancer recurrence, or decreased life expectancy.

Immunohistochemistry, FISH or a sandwich assay technique may also be used to measure the amount of MUC1 or MUC1-associated factor in which increased risk is associated with the measurement of MUC1 or MUC1-associated factor that is greater than 2 standard deviations above similar measurements on cells or tissues from a normal population, which indicates likelihood of acquiring drug resistance of a tumor or cancerous cells, cancer metastasis, cancer recurrence, or decreased life expectancy.

In the above method, when immunohistochemistry is used to measure the amount of MUC1 or MUC1-associated factor, membrane staining showing moderate (++) to strong (+++) readings in greater than 10% of a tumor specimen indicates the condition. The MUC1-associated factor may be MUC1*, NM23, MMP-14 or ADAM-17 (TACE).

The tumor or cancerous cell may be biliary tract cancer; bladder cancer; brain cancer; breast cancer; cervical cancer; choriocarcinoma; colon cancer; endometrial cancer; esophageal cancer; gastric cancer; hematological neoplasms; multiple myeloma; AIDS-associated leukemias and adult T-cell leukemia lymphoma; intraepithelial neoplasms; liver cancer; lung cancer; lymphomas; neuroblastomas; oral cancer; ovarian cancer; pancreatic cancer; prostate cancer; rectal cancer; sarcomas; skin cancer; testicular cancer; thyroid cancer; or renal cancer.

The invention is also directed to a method of determining a cancer patient's suitability for treatment with a MUC1-targeting therapy, comprising measuring an amount of MUC1 or MUC1-associated factor that a patient's cancer cells or tumor expresses, in which the MUC1 or MUC1-associated factor amount of ++ to +++ in more than 10% of the cancer cells sampled indicates that MU1-targeting therapy is appropriate.

The invention is also directed to the above methods, and further includes measuring an amount of HER2 in the cancerous cells or tumor, wherein immunohistochemistry staining for HER2 that is weak to moderate membrane staining in more than 10% of the tumor; or FISH ratio of HER2 expression relative to CEP17 expression, wherein a ratio of 2 or greater HER2:CEP17, indicates that treatment with a HER2-targeting agent and a MUC1*-targeting agent is appropriate.

The inventive method also includes carrying out immunohistochemistry staining for HER2 that is moderate to strong membrane staining in more than 30% of the tumor; or FISH ratio of HER2 expression relative to CEP17 expression, wherein a ratio of 3.0 or greater HER2:CEP17, indicates that treatment with a HER2-targeting agent and a MUC1*-targeting agent is appropriate.

The anti-cancer treatment using the MUC1-targeting therapy may further include treatment with Cisplatin, AraC, Etoposide, cyclophosphamide, taxol, or doxorubicin. Further, the method may include treatment with trastuzumab, Cisplatin, AraC, Etoposide, cyclophosphamide, taxol, or doxorubicin, which incorporates measuring an amount of MUC1* and an amount of HER2.

In another aspect, the invention is directed to a method for reducing breast cancer tumor size which cells present both MUC1* and HER2, comprising contacting the tumor with an effective amount of a HER2 disabling agent and a MUC1* disabling agent. HER2 disabling agent may be trastuzumab and the MUC1* disabling agent may be a monovalent anti-MUC1* antibody.

In particular, present invention is based on the discovery that cells that have acquired HERCEPTIN® resistance show a significant increase in the expression level of the cleaved form of the MUC1 protein, called MUC1*, which has been previously reported to function as a growth factor receptor. The therapeutic effect of HERCEPTIN® is completely restored if the resistant cancer cells are treated with HERCEPTIN® plus an agent that disables the MUC1* growth factor receptor. Additionally, HERCEPTIN® and MUC1* disabling agents appear to work synergistically; the growth of breast cancer cell lines that are intrinsically resistant to HERCEPTIN® are effectively inhibited by the combination of HERCEPTIN® and a MUC1* disabling agent. Further, cells that have acquired HERCEPTIN-resistance have also acquired resistance to standard chemo therapy agents, such as but not limited to taxol, cyclophosphamide and doxorubicin.

The therapeutic effect of these cancer drugs is completely restored if they are administered in combination with a MUC1* disabling agent. Patients at risk for developing early acquired drug resistance can be identified by assessing an amount of MUC1* presented on their cancer cells. Patients undergoing chemotherapy can be monitored for signs of acquiring drug resistance by measuring levels of shed MUC1 or NM23 in bodily fluids. Acquired HERCEPTIN® resistance can be avoided or reversed by treating with a combination therapy that includes HERCEPTIN® and a MUC1* disabling agent such as a monovalent MUC1* antibody or Fab.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 is a graph of cell growth plotted as a function of the concentration of either bivalent (by) or monovalent (mv) anti-MUC1* that is added to cells in culture. ZR75-30 is a MUC1-positive breast cancer cell line (ATCC) and K293 cells are MUC1-negative human embryonic kidney (HEK293) cells. Bivalent anti-MUC1* stimulates cell growth by dimerizing MUC1* receptors, whereas the monovalent antibody inhibits cell growth by blocking interaction of MUC1* with its native ligand. Neither antibody has any effect on MUC1-negative cells.

FIG. 2 shows Western blots tracking the phosphorylation state of ERK2 in response to treatment with either bivalent anti-MUC1* or monovalent anti-MUC1*. ERK2 phosphorylation is a key step in the activation of the MAP kinase cell proliferation signaling pathway. The upper blot shows that 10 minutes after the addition of bivalent anti-MUC1*, ERK2 is phosphorylated. The lower blot shows that the addition of the monovalent anti-MUC1* suppresses basal levels of ERK2 phosphorylation.

FIG. 3 shows bar graphs that measure cell death when MUC1-negative cells are transfected with MUC1, MUC1* or the empty vector (as a control), then treated with chemotherapy agents. A. MUC1-transfected HCT-116 (human colon cancer) cells, treated with cisplatin; B. MUC1-transfected 3Y1 (rat fibroblast) cells, treated with AraC; and C. MUC1-transfected HCT-116 (human colon cancer) cells, treated with Etoposide. MUC1*-9, MUC1*-10, MUC1-1-17, MUC1-44, MUC1-1-12, MUC1-1-18 or MUC1-10, MUC1-18, MUC1-8, MUC1-17, and MUC1-9, refer to single cell clone notation and MUC1 is the full-length protein while MUC1* is the construct that is comprised of the complete cytoplasmic tail and transmembrane domain of MUC1 but the extracellular domain is comprised only of the nat-PSMGFR sequence.

FIG. 4 is a graph of cell growth plotted as a function of HERCEPTIN® concentration for BT474 breast cancer cells and BTRes1 and BTRes2 which are BT474 cell pools that have acquired resistance to HERCEPTIN®.

FIG. 5 shows photos of Western blots of parent cell line BT474 cells compared to the HERCEPTIN® resistant pools, BTRes1 and BTRes2. Comparison is made between levels of MUC1*, MUC1 and HER2 before and after the cells acquired HERCEPTIN® resistance.

FIG. 6 is a graph of cell growth plotted as a function of HERCEPTIN® concentration for BT474 breast cancer cells and HERCEPTIN® resistant cells BTRes1 and BTRes2; an anti-MUC1* Fab or a control Fab were added to a final concentration of 2.5 ug/ml as indicated.

FIG. 7 is a graph of cell growth plotted as a function of HERCEPTIN® concentration for drug resistant cell pool BTRes1; the upper dotted trace is measured cell growth when HERCEPTIN® alone was added and the lower solid trace shows growth inhibition results when HERCEPTIN® and the small molecule MUC1 antagonist MN397 (pictured above) are added together.

FIG. 8 is a graph of cell growth plotted as a function of HERCEPTIN® concentration for drug resistant cell pool BTRes1 (upper solid trace), for parent cell line BT474 transfected with a control siRNA (middle dotted trace) and for drug resistant cell pool BTRes1 transfected with a MUC1-specific siRNA (lower dashed trace). Inset is a photo of a Western blot in which the gel is probed with an anti-MUC1* antibody and shows that MUC1* has been effectively downregulated by the siRNA.

FIG. 9A is a bar graph comparing the amount of drug-induced cell death for BT474 cells (striped bars) and HERCEPTIN-resistant BTRes1 cells (solid bars) in response to treatment with the cytotoxic cancer drug, taxol. The bars on the left represent the measured cell death for cells treated with taxol and a control Fab. The bars on the right represent the measured cell death for cells treated with taxol plus an anti-MUC1* Fab. The results show that multi-drug resistance is overcome by disabling MUC1*. B is a bar graph showing that the cytotoxic effect (BT474 striped bars) of chemotherapy drug Cyclophosphamide is lost when cells acquire HERCEPTIN® resistance (solid & hatched bars on left). The killing effect of the drug is restored when drug resistant cells are treated with both an anti-MUC1* Fab and Cyclophosphamide (right). C is a bar graph showing that the cytotoxic effect (BT474 striped bars) of chemotherapy drug Doxirubicin is lost when cells acquire HERCEPTIN® resistance (solid & hatched bars on left). The killing effect of the drug is restored when drug resistant cells are treated with both an anti-MUC1* Fab and Doxirubicin (right).

FIG. 10A is a graph that plots the growth of T47D breast cancer cells as a function of HERCEPTIN® added in the presence or absence of an anti-MUC1* Fab. T47D cell are characterized by very high expression of MUC1* and have been reported to be unaffected by HERCEPTIN® treatment. The graph shows that T47D cell growth is inhibited by HERCEPTIN® when added in combination with a MUC1*-disabling agent. B. breast cancer cell line ZR-75-30, also a high expresser of MUC1*, is inhibited by the combination of anti-MUC1* Fab and HERCEPTIN® in a dose dependent manner. C. T47D cells become HERCEPTIN® sensitive when treated with HERCEPTIN® and siRNA that downregulates MUC1* expression.

FIGS. 11A-11D show four (4) photographs of human breast cancer specimens under magnification. (A) and (C) are adjacent slices from the same section of a MUC1-positive cancer and (B) and (D) are adjacent slices from the same section of a MUC1-negative cancer. Sections (A) and (B) (top) have been treated with anti-PSMGFR that binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining. This result indicates that the predominant form of the MUC1 receptor on the surface of cancer cells is devoid of the tandem repeat portion and is comprised essentially of the PSMGFR sequence.

FIGS. 12A-12C show three (3) photographs of adjacent slices of a breast cancer biopsy specimen stained with either A) H&E; B) anti-PSMGFR, or C) VU4H5. Comparison of B) and C) show that VU4H5 stains the cytoplasm diffusely while anti-PSMGFR clearly stains the cell surface membrane. This indicates that, on cancer cells, the MUC1 receptor has been cleaved to release the tandem repeat portion but leaves the portion containing the PSMGFR sequence attached to the cell surface.

FIGS. 13A-13D show four (4) photographs of human lung cancer tissue specimens under magnification. (A) and (C) are adjacent slices from a first section of a MUC1-positive lung cancer and (B) and (D) are adjacent slices from a MUC1-negative cancer. Sections (A) and (B) (top) have been treated with anti-PSMGFR, which binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining and that anti-PSMGFR staining is restricted to the cell surface. These results again indicate that the predominant form of the MUC1 receptor on the surface of MUC1-positive lung cancer cells is mostly devoid of the tandem repeat portion and is comprised essentially of the PSMGFR sequence.

FIGS. 14A-14C show the same set of MUC1-positive lung cancer tissue specimens as in FIGS. 13A-13D at a greater magnification. At enhanced magnification, it is readily observed that the anti-PSMGFR staining is restricted to the cell surface whereas VU4H5 is diffuse and cytoplasmic, confirming that the MUC1 receptor on the surface of MUC1-positive lung cancer cells is cleaved to release the tandem repeat domain and leave the MGFR portion attached to the cell surface.

FIGS. 15A-15B show two (2) photographs of colon cancer tissue specimens that have been stained with either (A) anti-PSMGFR or (B) VU4H5. The arrows point to portions of the section that are very cancerous as indicated by the fact that they have lost all cellular architecture. Section (A), shows dark regions of staining with anti-PSMGFR but the same region of the adjacent section (B), which has been stained with VU4H5, which recognizes the tandem repeat portion of the MUC1 receptor, shows no staining at all. These results indicate that, in MUC1-positive colon cancer, the MUC1 receptor has been cleaved to release the tandem repeat portion but leaves the portion of the receptor that contains the PSMGFR sequence intact and attached to the cell surface.

FIG. 16A shows cross section of a normal fallopian tube tissue in which MUC1* expression is limited to the luminal edge of the duct; whereas 16B shows cancerous breast tissue in which MUC1* is uniformly expressed over the entire tissue and is not restricted to the luminal edge.

FIG. 17 shows that human breast cancer stem cells, defined as CD44hi/CD24lo, have higher expression of MUC1* and MUC1 than the original mixed cancer cell population from which they were isolated. The red fluorescence is anti-MUC1* and the green fluorescence is VU4H5. Both sets of images were taken at the same exposure.

FIG. 18 is a table that summarizes the results of probing a variety of human tissue specimens with either anti-MUC1* or VU4H5 which recognizes full-length MUC1. Included are pathologist's score and characterizations.

### DETAILED DESCRIPTION OF THE PREFERRED EMBODIMENTS

In the present application, “a” and “an” are used to refer to both single and a plurality of objects.

The term “MUC1 Growth Factor Receptor” (MGFR) is a functional definition meaning that portion of the MUC1 receptor that interacts with an activating ligand, such as a growth factor or a modifying enzyme such as a cleavage enzyme, to promote cell proliferation. The MGFR region of MUC1 is that extracellular portion that is closest to the cell surface and is defined by most or all of the PSMGFR, as defined below. The MGFR is inclusive of both unmodified peptides and peptides that have undergone enzyme modifications, such as, for example, phosphorylation, glycosylation, etc. Results of the invention are consistent with a mechanism in which this portion is made accessible to the ligand upon MUC1 cleavage at a site associated with tumorigenesis that causes release of the some or all of the IBR from the cell. MGFR is also known as MUC1*.

As used herein, “anti-PSMGFR” refers to any antibody that recognizes a region of the MGFR and optionally any portion of PSMGFR. Antibody to nat-PSMGFR is exemplified and preferred in the application, but is not meant to be limited to an antibody made against this specific sequence, as other fragments of MGFR and PSMGFR are also contemplated.

The term “Primary Sequence of the MUC1 Growth Factor Receptor” (PSMGFR) is a peptide sequence that defines most or all of the MGFR in some cases, and functional variants and fragments of the peptide sequence, as defined below. The PSMGFR is defined as SEQ ID NO:10 listed below in Table 1, and all functional variants and fragments thereof having any integer value of amino acid substitutions up to 20 (i.e. 1, 2, 3, 4, 5, 6, 7, 8, 9, 10, 11, 12, 13, 14, 15, 16, 17, 18, 19, or 20) and/or any integer value of amino acid additions or deletions up to 20 at its N-terminus and/or C-terminus. A “functional variant or fragment” in the above context refers to such variant or fragment having the ability to specifically bind to, or otherwise specifically interact with, ligands that specifically bind to, or otherwise specifically interact with, the peptide of SEQ ID NO:10. One example of a PSMGFR that is a functional variant of the PSMGFR peptide of SEQ NO: 10 (referred to as nat-PSMGFR—for “native”) is SEQ NO: 12 (referred to as var-PSMGFR), which differs from nat-PSMGFR by including an -SPY- sequence instead of the native -SRY- (see bold text in sequence listings). Var-PSMGFR may have enhanced conformational stability, when compared to the native form, which may be important for certain applications such as for antibody production. The PSMGFR is inclusive of both unmodified peptides and peptides that have undergone enzyme modifications, such as, for example, phosphorylation, glycosylation, etc.

The term “Tumor-Specific Extended Sequence of the MUC1 Growth Factor Receptor” (TSESMGFR) is a peptide sequence (See, as an example, Table 1—SEQ ID NO:16) that defines a MUC1 cleavage product found in tumor cells that remains attached to the cell surface and is able to interact with activating ligands in a manner similar to the PSMGFR.

As used herein, “multimerization” of the receptors includes without limitation dimerization of the receptors. Further, multimerization includes binding of co-receptor or co-receptors with MUC1, or binding of multiple MUC1 receptors with each other, which may be gathered together by a ligand or ligands possessing multiple valences.

A “ligand” to a cell surface receptor, refers to any substance that can interact with the receptor to temporarily or permanently alter its structure and/or function. Examples include, but are not limited to binding partners of the receptor, (e.g. antibodies or antigen-binding fragments thereof), and agents able to alter the chemical structure of the receptor (e.g. modifying enzymes).

A “growth factor” refers to a species that may or may not fall into a class of previously-identified growth factors, but which acts as a growth factor in that it acts as an activating ligand.

The term “cancer”, as used herein, may include but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Preferred cancers are; breast, prostate, lung, ovarian, colorectal, and brain cancer.

The term “cancer treatment” as described herein, may include but is not limited to: chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment.

An “agent for prevention of cancer or tumorigenesis” means any agent that counteracts any process associated with cancer or tumorigenesis described herein.

An “agent that enhances cleavage of a cell surface receptor interchain binding region” as used herein is any composition that promotes cleavage at a particular location by modifying MUC1 with sugar groups or phosphates that create a recognition motif for cleavage at that location. Other enzymes can promote cleavage of receptors by activating other cleavage enzymes. One way to select agents that enhance cleavage of a cell surface receptor IBR is to first identify enzymes that affect cleavage as described above, and screen agents, and their analogs, for their ability to alter the activity of those enzymes. Another way is to test agents that are known to affect the activity of similar enzymes (e.g. from the same family) for their ability to alter the site of cleavage of MUC1, and to similarly test analogs of these agents. Alternatively, agents are screened in a cell-free assay containing the enzyme and MUC1 receptors, and the rate or position of cleavage measured by antibody probing, Polymerase Chain Reaction (PCR), or the like. Alternatively, without first identifying enzymes that affect MUC1, agents are screened against cells that present MUC1 for the agents' ability to alter cleavage site or the rate of cleavage of MUC1. For example, agents can be screened in an assay containing whole cells that present MUC1 and aggregation potential of the cell supernatant can be measured, an indication of the amount of IBR that remains attached to the cleaved portion of MUC1, i.e. the degree of cleavage between MGFR and IBR. In another technique, agents can be screened in an assay containing whole cells that present MUC1, the supernatant removed, and the cell remain tested for accessibility of the MGFR portion, e.g. using a labeled antibody to the MGFR. Agents can be identified from commercially available sources such as molecular libraries, or rationally designed based on known agents having the same functional capacity and tested for activity using the screening assays.

A subject, as used herein, refers to any mammal (preferably, a human), and preferably a mammal that has a disease that may be treated by administering the inventive composition to a site within the subject. Examples include a human, non-human primate, cow, horse, pig, sheep, goat, dog, or cat. Generally, the invention is directed toward use with humans.

The samples used herein are any body tissue or body fluid sample obtained from a subject. Preferred are body fluids, for example lymph, saliva, blood, urine, milk and breast secretions, and the like. Blood is most preferred. Samples of tissue and/or cells for use in the various methods described herein can be obtained through standard methods including, but not limited to: tissue biopsy, including punch biopsy and cell scraping, needle biopsy, and collection of blood or other bodily fluids by aspiration or other methods.

As used herein, “MUC1-associated factor” refers to biological molecules that directly or indirectly associate with MUC1 or MUC1*. For example, NM23, the native ligand of MUC1* is a MUC1-associated factor; cleavage enzymes MMP-14 and TACE (ADAM-17) are MUC1-associated factors; portions of the MUC1 protein that are shed from the cell surface following cleavage and which in some cases can be detected in bodily fluids are MUC1-associated factors.

As used herein, “MUC1 positive” cell refers to a cell that expresses the MUC1 gene or gene product.

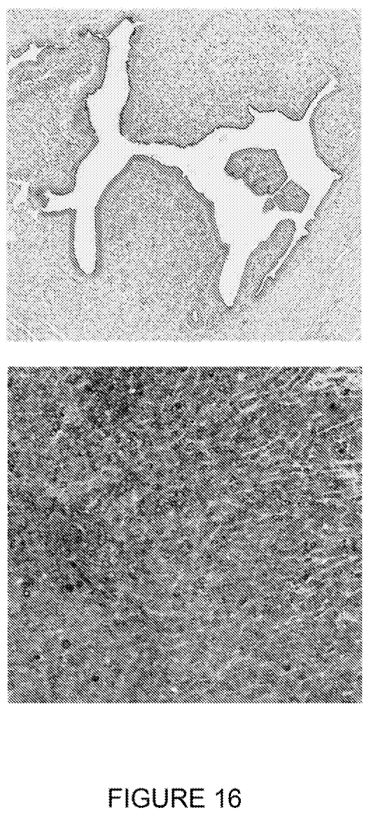

As used herein, a “MUC1-positive cancer” or a “MUC1*-positive cancer” refers to a cancer that is characterized by the aberrant expression of MUC1, wherein aberrant may refer to the overexpression of the MUC1 gene or gene product, or the loss of the normal expression pattern of MUC1 or MUC1* which, in the healthy state, is restricted to the apical border of the cell or the luminal edge of a duct or an increase in the amount of MUC1 that is cleaved and shed from the cell surface. FIG. 16 illustrates this point. FIG. 16A shows cross section of a normal fallopian tube tissue in which MUC1* expression is limited to the luminal edge of the duct; whereas FIG. 16B shows cancerous breast tissue in which MUC1* is uniformly expressed over the entire tissue and is not restricted to the luminal edge.

As used herein, “normal” cells are grown anchorage-independently when MUC1* receptor is transfected into the cell. At certain receptor densities (it appears high densities self-signal), anchorage independent cell growth proceeds without stimulation; the addition of a bivalent anti-MUC1* antibody or other multimerizing agent, preferably a dimerizing agent, enhances this ability to grow anchorage-independently.

As used herein, trastuzumab (more commonly known under the trade name HERCEPTIN®) is a humanized monoclonal antibody that acts on the HER2/neu (erbB2) receptor. Trastuzumab's principal use is as an anti-cancer therapy in breast cancer in patients whose tumors over-express (that is, “produce more than the usual amount of”) this receptor. In the present application, Trastuzumab and HERCEPTIN® are used interchangeably. Occasionally herein, HERCEPTIN® may be used without the trademark symbol ®, however, it is understood that HERCEPTIN® is a registered trademark.

MUC1 Expression in Tumor Cells

The inventors previously disclosed that a form of the MUC1 receptor that has a shortened extracellular domain is highly expressed on cancer cells and that it is the growth factor receptor activity of this truncated MUC1 that drives the growth of cancer cells. The extracellular domain of the truncated MUC1 consists primarily of the PSMGFR sequence, as shown in Table 1 (SEQ ID NO:10 or SEQ ID NO:11, but has the transmembrane and cytoplasmic domains of MUC1 and is referred to herein as MUC1* and also as MGFR. The shortened form of MUC1 receptor is most often the result of a cleavage event. However, MUC1 variants with truncated extracellular domains, such as MUC1/Y, can also be produced by alternative splicing and the like.

It has been estimated that MUC1 is aberrantly expressed on 75% of all human solid tumors and may exist in other types of cancer as well. In this context, aberrant expression has historically referred to the observation that on healthy epithelium the receptor is clustered at the apical border while on cancer cells, the receptor is uniformly distributed over the entire cell surface. It has also been known for some time that a portion of the receptor can be detected in the blood of late stage breast cancer patients. In addition, there were reports in the literature describing possible cleavage sites in the MUC1 extracellular domain.

To establish a possible link between cancer and MUC1 cleavage and to determine which MUC1 species was expressed on the surface of cancer cells, we probed a panel of human cancerous tissue specimens with two antibodies: one that recognized a portion of the receptor that should be released from the cell surface after receptor cleavage and another that recognized a portion that should remain attached to the cell surface. The first antibody is a rabbit polyclonal antibody raised against the var-PSMGFR (SEQ ID NO:12), referred to herein as anti-PSMGFR and also as anti-MUC1*. The second antibody is a commercially available antibody (VU4H5) that binds to the tandem repeats of the MUC1 receptor that are at N-terminal end of the receptor and distal to the cell surface. It should be noted that the MUC1 receptor contains hundreds of the tandem repeat motifs, so that each full-length receptor will be bound by hundreds of VU4H5 antibodies. In sharp contrast, the sequence to which anti-MUC1* binds occurs only once per receptor.

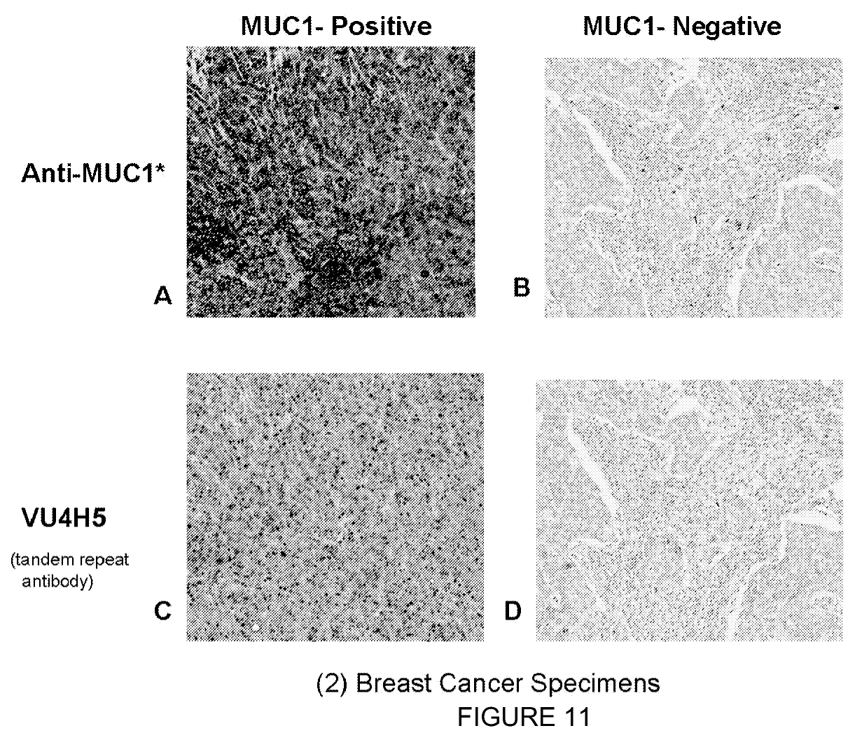

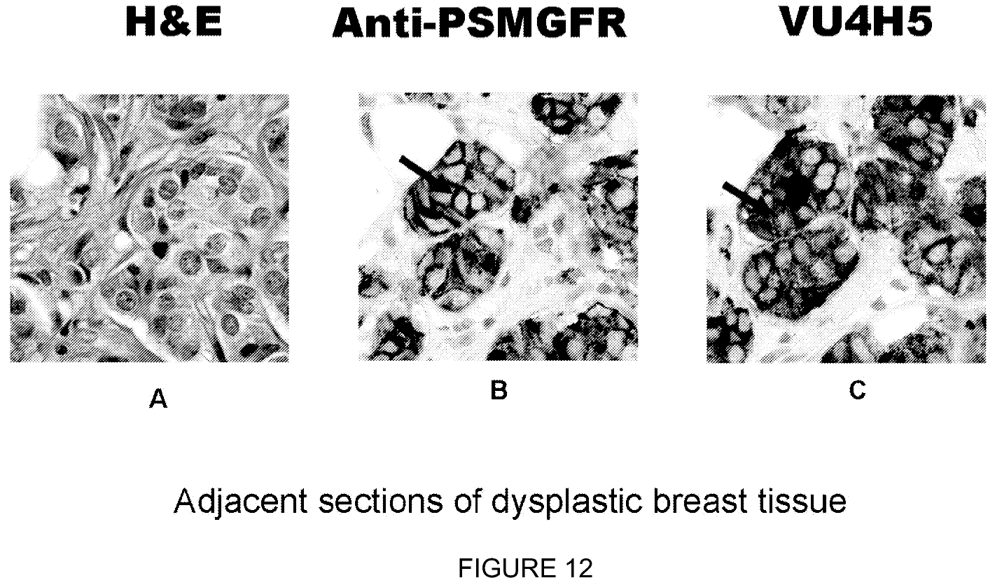

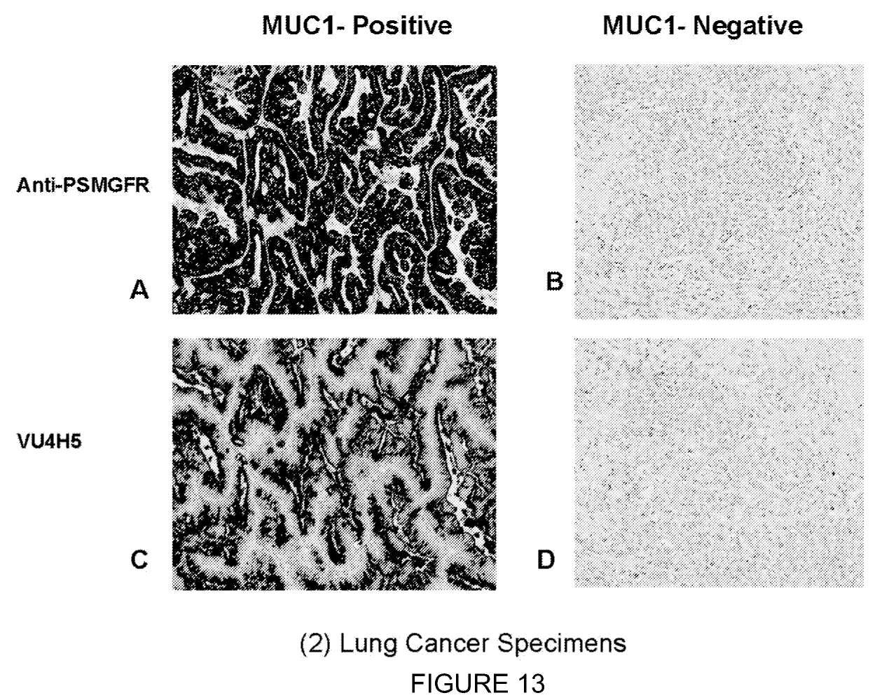

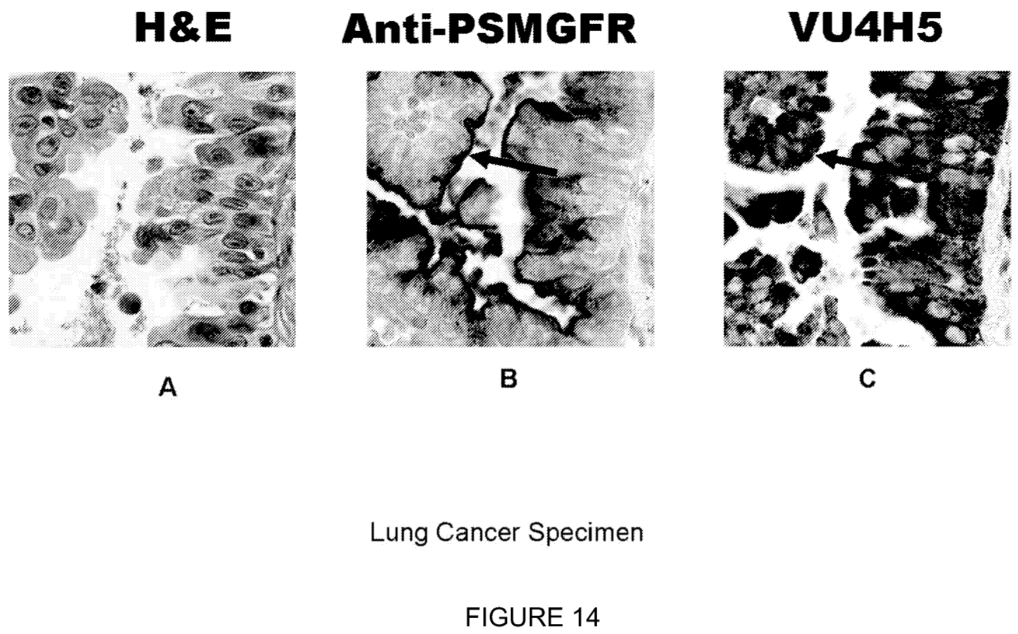

FIGS. 11-15 are human cancerous tissue specimens. The dual antibody staining experiment shows that most of the MUC1 on cancerous tissue has been cleaved to release the tandem repeat portion and leaves the MGFR (MUC1*) portion attached to the cell surface. The predominant MUC1 species expressed on cancer cells reacts with anti-PSMGFR but not with VU4H5. FIG. 11 shows four (4) photographs of human breast cancer specimens under magnification. (A) and (C) are adjacent slices from the same section of a MUC1-positive cancer and (B) and (D) are adjacent slices from the same section of a MUC1-negative cancer. Sections (A) and (B) (top) have been treated with anti-PSMGFR. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining. This result indicates that the predominant form of the MUC1 receptor on the surface of cancer cells is devoid of the tandem repeat portion and is comprised essentially of the PSMGFR sequence. FIG. 13 shows four (4) photographs of human lung cancer tissue specimens under magnification. (A) and (C) are adjacent slices from a first section of a MUC1-positive lung cancer and (B) and (D) are adjacent slices from a MUC1-negative cancer. Sections (A) and (B) (top) have been treated with anti-PSMGFR, which binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells.

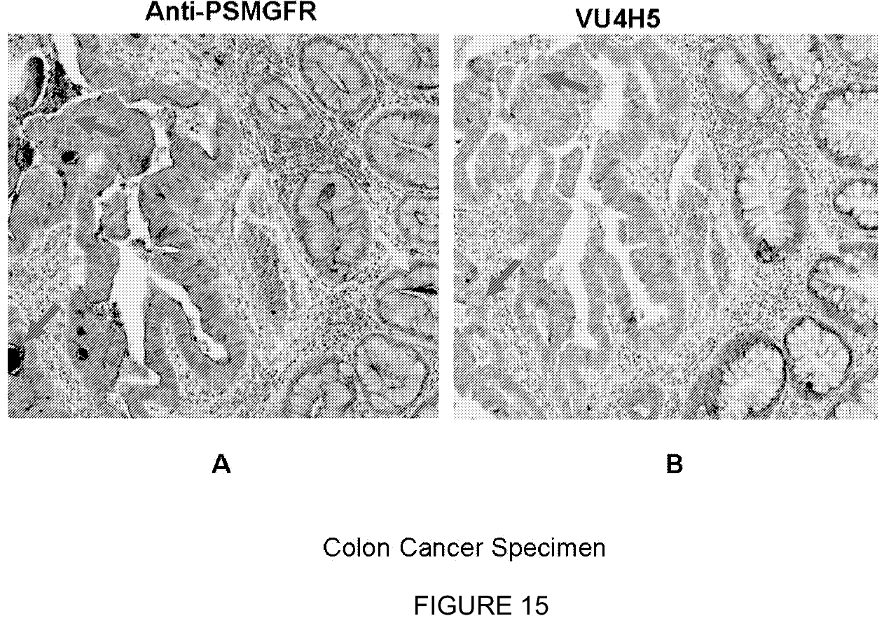

Note the greater intensity of the anti-PSMGFR staining compared to VU4H5 staining and that anti-PSMGFR staining is restricted to the cell surface. Note also that there is heavy cytoplasmic staining for MUC1 and no surface staining, when probing with VU4H5 (FIGS. 11C, 12C, and 14C). However, probing of the adjacent tissue slice with anti-PSMGFR showed that the entire cell surface was uniformly coated with a cleaved MUC1 that did not contain the tandem repeat section but did contain the PSMGFR sequence (FIGS. 11A, 12B, and 14B). These results again indicate that the predominant form of the MUC1 receptor on the surface of MUC1-positive lung cancer cells is mostly devoid of the tandem repeat portion and is comprised essentially of the PSMGFR sequence. FIG. 15 shows two (2) photographs of colon cancer tissue specimens that have been stained with either (A) anti-PSMGFR or (B) VU4H5. The arrows point to portions of the section that are very cancerous as indicated by the fact that they have lost all cellular architecture. Section (A), shows dark regions of staining with anti-PSMGFR but the same region of the adjacent section (B), which has been stained with VU4H5, which recognizes the tandem repeat portion of the MUC1 receptor, shows no staining at all. These results indicate that, the fastest growing portions of the tumor present a form of MUC1 that is devoid of the tandem repeat portion but leaves the portion of the receptor that contains the nat-PSMGFR sequence intact and attached to the cell surface. The results of these staining experiments show that the major MUC1 species on the surface of human cancerous tissue is MUC1*, while there is a lesser amount of full-length MUC1 on the cell surface, implying a high degree of MUC1 receptor cleavage in cancer.

To determine whether cultured cancer cell lines displayed ratios of cleaved MUC1 to uncleaved MUC1 that were similar to the ratio observed on cancerous tissue specimens, we analyzed MUC1-positive tumor cell lysates by SDS-PAGE. As we had observed for the cancerous tissue specimens, the vast majority of the MUC1 receptors on cultured tumor cell lines have been cleaved to leave the MUC1* portion attached to the cell surface, while a smaller percentage of the receptors includes the tandem repeat portion. Human-derived cancer cell lines that are known to have high expression levels of MUC1 were subjected to western blot analysis A low percentage acrylamide gel (6%) showed a high molecular weight protein band at about 220 kDa that reacted with the VU4H5 antibody. A high percentage gel (12%) showed a low molecular weight species that ran with an apparent molecular weight between 20 and 30 kDa that reacted with the anti-PSMGFR antibody. These data taken together with data presented above indicate that both cultured cancer cell lines and human cancerous tissue display high expression of a low molecular weight species that reacts with an antibody raised against the PSMGFR sequence.

A key mechanism of cell growth in MUC1 positive cancers may depend more on the amount of MUC1 cleavage that occurs rather than the overall amount of MUC1 receptor that is expressed. Low molecular weight species that migrate on an acrylamide gel with an apparent molecular weight of around 20-30 kD (some glycosylated) exist in MUC1-positive tumor cells but do not exist in sufficient numbers to be detectable in non-tumor MUC1 cells. Two cleavage sites of the MUC1 receptor in tumor cells were previously identified. The first cleavage site occurs in the middle of the IBR and the second cleavage site, which our evidence indicates is the more tumorigenic form, occurs at the C-terminal end of the IBR: the first cleavage site being located at the N-terminus of TPSIBR (SEQ ID NO:17) and the second cleavage site being located at the N-terminus of the nat-PSMGFR having SEQ ID NO:13. When cleavage occurs at the first site, the portion of the receptor that remains attached to the cell surface is similar to TSESMGFR (See Table 1, SEQ ID NO:16, but with the native SRY sequence). When cleaved at the second site, the remaining portion is a PSMGFR as shown in Table 1, SEQ ID NO:11. This low molecular weight species that is tumor specific consists essentially of the native PSMGFR sequence and in some cases the TSESMGFR sequence and is available to cognate ligands, i.e. not self-aggregated, than on the overall amount of MUC1 receptor expressed by the cell. Supporting this conclusion, susceptibility of tumor cells to proliferate was found, within the context of the present invention, to be a function of the amount of the shorter form of the MUC1 receptor.

The portion of the MUC1 receptor that acts as a growth factor receptor is a cleavage product in which much or all of the IBR is released from the cell surface. Further, these results support the conclusion that tumors in which a good percentage of the MUC1 receptors have been cleaved to release the TPSIBR (SEQ ID NO:18) are especially aggressive cancers and those that are cleaved to release the entire IBR, leaving PSMGFR (SEQ ID NO:11) attached to the cell surface are even more aggressive. Therefore, antibodies that are raised against the TPSIBR (SEQ ID NO:18) portion of the MUC1 receptor can be used to assess the aggressiveness of cancers that are MUC1-positive.

Cleavage enzymes MMP14 and ADAM17 cleave MUC1 in cancer cells

MMP14 and ADAM17 exist on the surface of the same tumor cells that express MUC1*. This leads to the conclusion that tumor cells, the MUC1 receptor is cleaved to leave MUC1* on the cell surface and this cleavage is carried out by the membrane matrix metallo protease MMP14 (also called MT1-MMP and ADAM17).

Cancer Stem Cell Identification and Isolation

It has recently been established that there are cancer stem cells as defined by the ability to confer disease in a host with the introduction of small numbers of cancer cells on the order of tens or hundreds rather than the typical requirement for millions of cells. There are of course many therapeutic reasons for wanting to detect cancer stem cells in a variety of circumstances. The art of identifying cancer stem cells is in its infancy. Current research shows that cancer stem cells are characterized by a set of markers that includes CD133, CD44 and ESA. Recall that we have demonstrated that MUC1* is highly expressed on the surface of cancer cells and that a high level of surface expression of MUC1*, together with a low level of surface expression of full-length MUC1, indicates aggressive cancer cell growth that is also resistant to treatment with chemotherapy agents. Therefore, an improvement in the current method for identifying cancer stem cells is to select cells that express known cancer markers, such as CD133, CD44 and ESA, but also must display high levels of surface staining of MUC1* and low or no staining of the repeat portions of the MUC1 receptor.

Methods of the invention for identifying and isolating cancer cells and cancer stem cells “CSCs” are employed in a variety of circumstances. For example, CSCs are identified and removed from a patient as a part of a therapeutic regimen. Since CSCs can be in the circulation, it would be advantageous to use methods of the invention to cleanse a patient's bodily fluids and other substances, including blood, bone, and bone marrow of CSCs before subsequent re-introduction into the patient or into another patient. Re-introduction of cells may take place after several types of therapeutic interventions to alleviate the cancer, which may include chemotherapy and radiation therapy. In one embodiment, methods of the invention are used to identify, isolate and remove cancer stem cells from a patient's blood as an anti-cancer therapy, a cancer preventative or cancer recurrence preventative. The patient's blood is removed from the patient and passed through an instrument that has a chamber that presents antibodies against MUC1* and a panel of other CSC markers such as CD144, CD44, and/or ESA. As MUC1-1-positive cells pass through this portion of the instrument, they are captured and retained within the instrument while the sanitized blood is re-introduced into the patient. Methods of the invention are used for identification and isolation of cancer stem cells and subsequent removal of stem cells or cancer stem cells from a patient, for example to sanitize (to ensure the removal of all cancer stem cells from a patient) a patient's blood or bone marrow after cancer surgery or treatment.

The invention further anticipates using methods described herein to identify and isolate cancer stem cells from a variety of sources, for the purpose of ridding a person, animal or person/animal derived materials of cancer stem cells. Instances in which it is desired to rid biological materials of cancer and CSCs include those cases wherein material is destined for transplant or transfusion. Materials and/or sources from which such cancer stem cells may be identified and/or isolated from include but are not limited to a cancer patient, person suspected of having cancer, a person living or deceased, donating or being considered for receiving transplant substances including organs, cornea, tissue, skin, bone, bone marrow, and the like. Substances destined to be introduced into live tissues, organs or persons by transplantation, transfusion, injection, or oral administration, wherein it would be desirable to rid the substances of cancer or cancer stem cells include blood, organs, tissues, skin, fat, stem cells, bone marrow and cartilage.

NM23

Activation of the MUC1* growth factor receptor is an autocrine process. Cells that express the growth factor receptor form of the MUC1 receptor, MUC1*, also produce the ligand that activates the receptor. The antibody stimulation experiments demonstrated that dimerization of the MUC1* receptor stimulates cell growth by triggering the MAP kinase cell proliferation cascade. NM23 is the ligand that dimerizes and activates the MUC1* growth factor receptor. Expression of the MUC1 receptor stimulates the expression of NM23. NM23 stimulation of MUC1* is self-regulated by an autocrine feedback loop. NM23 can exist as a monomer, dimer, tetramer or hexamer (reviewed in Lascu, et al. J. Bioenerg. Biomemb 2000 32(3):227-36). The NM23 dimer activates cell growth by dimerizing the MUC1* receptors. Higher order multimers of NM23, such as the hexamers, inhibit MUC1-dependent cell growth, by inducing clustering of the MUC1 receptors. Cancer or unchecked cell growth can be caused by a defect in this self-regulation of the MUC1 receptor. Mutations in NM23 that inhibit the formation of the protective hexamers can result in cancers. These mutations that inhibit hexamer formation show increased levels of NM23 dimers, which activate MUC1-dependent cell growth by dimerizing the MUC1* receptor. The mutant in NM23-H1, S120G is common in neuroblastoma, and it has been shown that the S120G mutation results in the reduction of hexamers and an increase in dimers in solution when compared to wild type protein (Kim, et al. Biochem Biophys Res Comm 2003 307: 281-9). Transfection of wild type NM23-H1, or mutants that did not affect hexamer formation inhibited migration of prostate cancer cells (Kim, et al. Biochem Biophys Res Comm 2003 307: 281-9) and breast cancer cells (MacDonald, et al. J Biol Chem 1996 271(41): 25107-16) in in vitro invasiveness assays, while transfection of mutant NM23-H1 constructs that harbored the S120G mutation did not inhibit invasiveness, and in some experiments, enhanced invasiveness (MacDonald, et al. J Biol Chem 1996 271(41): 25107-16).

A review of the current state of knowledge in this area shows the level of confusion that exists regarding the role of NM23 in differentiation. Okabe-Kado, et al (Cancer Research 1985 45: 4848-52) first identified an inhibitory factor of differentiation (as defined by the induction of phagocytic activity post-treatment of cells with dexamethasone) of murine myeloid leukemia M1 cells contained within the cultured medium of those cells. In a following paper (Okabe-Kado, et al, BBRC 1992 182(3):987-94), the inhibitory factor was identified as murine NM23-H2. Further work demonstrated the inhibition of erythroid differentiation of the erythroid leukemia cell lines K562, HEL and KU812F cells (Okabe-Kado, et al. Biochimica Biophysica Acta 1995 1267:101-106). In these experiments, erythrocytic differentiation was defined as positive staining for benzidine, and differentiation was induced by TGF-β1, Hemin and Retinoic Acid.

These experiments also demonstrate that recombinant human NM23-H1 and -H2, in the form of GST fusions block erythroid differentiation of HEL cells by TGF-β1, and that kinase-deficient human NM23-H2, and truncated NM23-H2 (aa1-60) are also capable of inhibiting differentiation of HEL cells by TGF-β1. Similar work done by this group around the same time (Okabe-Kado, et al FEBS Letters 1995 363: 311-315), showed that recombinant GST fusions of mouse, human, and rat NM23 1 and 2 proteins inhibited inhibition of differentiation of murine myeloid leukemia M1 cells, as defined by the induction of phagocytic activity post-treatment of cells with dexamethasone, and that the kinase activity of NM23-H2 is not required for this activity, and that N-terminal regions of NM23-H2 (aa1-60 and aa1-108) contain wild-type inhibitory activities.

NM23 can exist as a monomer, dimer, tetramer or hexamer (reviewed in Lascu, et al. J. Bioenerg. Biomemb 2000 32(3):227-36). Our evidence is that as a dimer, NM23 dimerizes the MUC1* receptor and triggers cell growth. At higher multimerization states, e.g. hexamers, they no longer dimerize the receptor to trigger cell growth or stave off differentiation. To the contrary, they inhibit both these activities by re-clustering the MUC1 receptors and inhibiting its growth factor receptor activity. Recombinant NM23 that may be in any one of four multimerization states, wherein the monomer, dimer (activates growth and delays differentiation) and hexamer (inhibits growth and induces differentiation) produce vastly different results are also seen.

Similarly, a suitable treatment for patients suffering from MUC1-positive cancers consists of providing NM23 that is able to form hexamers, either through direct administration or via a gene therapy approach.

MUC1* is a Predictor of Early Drug Resistance

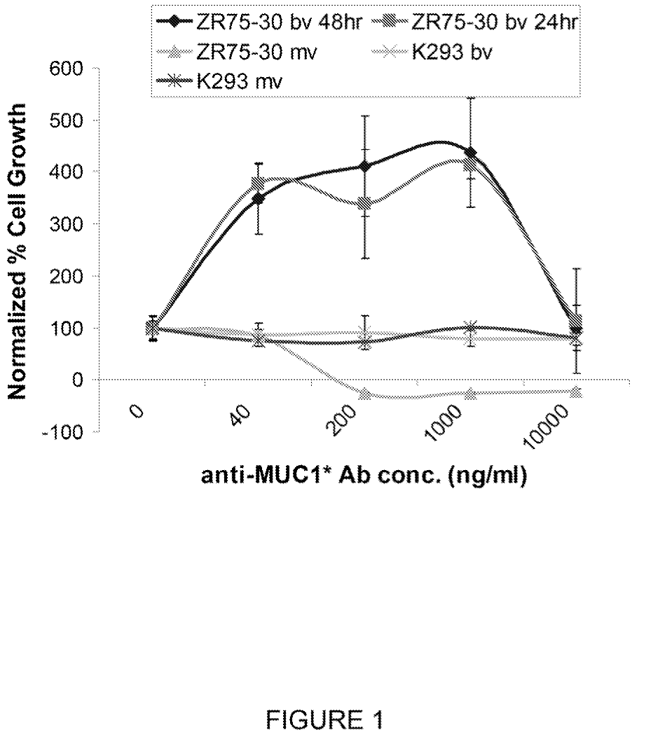

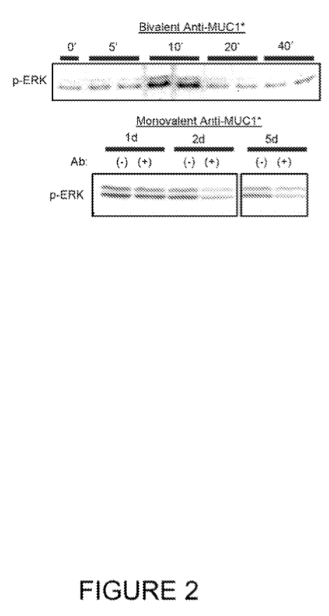

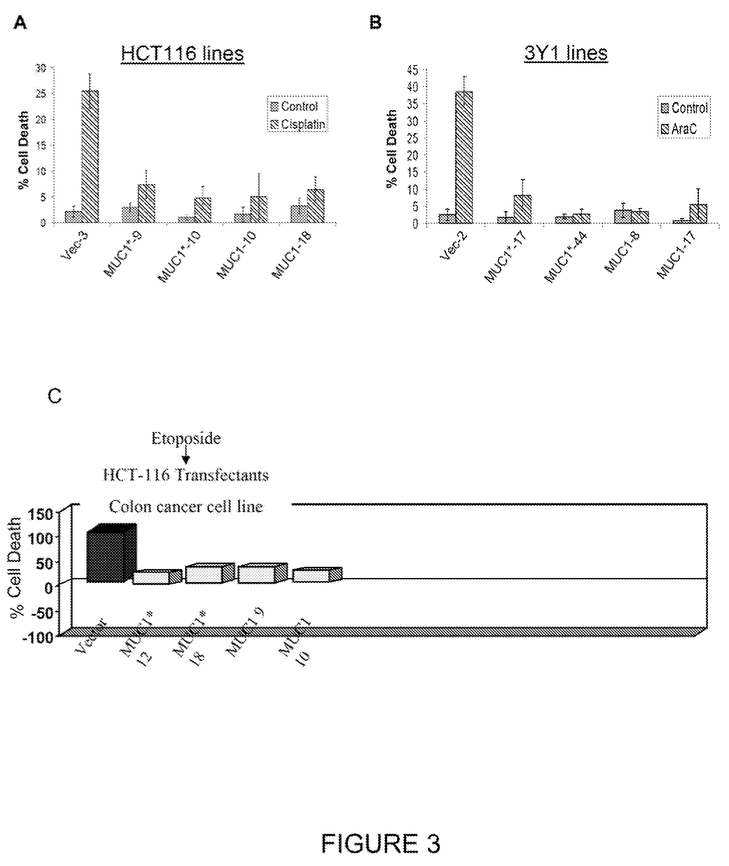

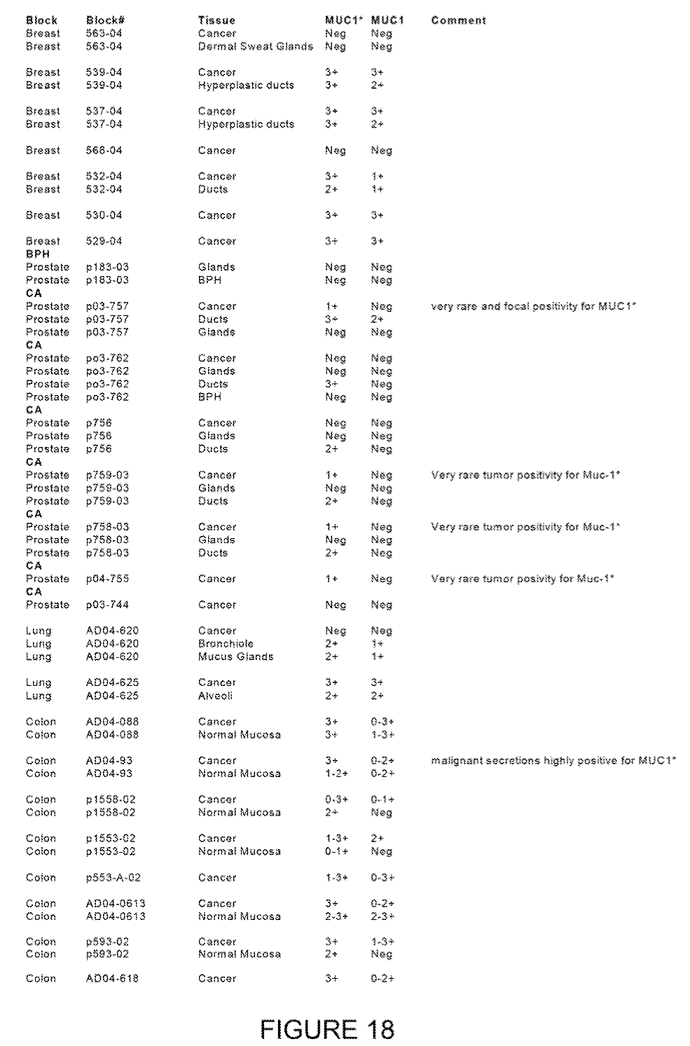

MUC1* is a proteolyzed cell surface receptor that functions as a growth factor receptor and drives cell proliferation, see FIG. 1. Dimerization of the PSMGFR region of the MUC1 receptor results in increased cell proliferation mediated at least in part by the MAP kinase signaling pathway. Further, we have shown that the dimeric form of NM23 is a dimerizing ligand of MUC1. Binding of NM23 dimers activates the growth factor receptor function of MUC1 and in particular, the cleaved form of MUC1, which we call MUC1*. Ligand-induced MUC1* dimerization leads to ERK2 phosphorylation and triggers cell division, see FIG. 2. Another way that MUC1* functions is through a survival mechanism. When MUC1* is transfected into MUC1-negative cells, the cells become resistant to cell death induced by standard chemo therapy agents like taxol, cyclophosphamide, doxorubicin and the like, see FIG. 3. Evidence indicates that cancer cells may develop a dependence on a particular receptor to drive their cell growth. However, the population of cancer cells in a tumor has subpopulations of cells that display a heterogeneous collection of cell surface receptors. After prolonged treatment with a drug that disables the predominant receptor, those cells either die out or do not replicate as quickly as other cells that do not depend on that receptor for growth. In this case, the makeup of the cancerous cell population is changed when non-targeted cells survive and takeover the population. Drug resistance can also happen when cells, in response to treatment with one growth factor receptor-targeting drug, upregulate a second growth factor receptor. Xenografted tumors in mice, for example, shrink or become stable for some period of time in response to HERCEPTIN® or other chemo therapy treatment, but then often become resistant to the drug and display an exponential increase in tumor growth. This quiescent period followed by accelerated tumor growth is characteristic of a tumor acquiring drug resistance when the cells shift to growth supported by a different non-targeted growth factor receptor. Drug resistance can also result when the therapy kills off routine cancer cells but enriches the population with cancer stem cells, which are not easily eradicated by standard chemotherapy drug like taxol, cisplatin, doxorubicin and the like. We have shown that cancer stem cells have a much higher concentration of MUC11 than the original cell population from which they were selected, see FIG. 18.

HERCEPTIN® Resistance as a Model System for Cancer Drug Resistance

An increase in the amount of MUC1 or MUC1 that a patient's cancer cells produce accompanies the acquisition of cancer drug resistance. An abundance of MUC1 or MUC1* predicts the early acquisition of drug resistance. For instance, cells that are treated with levels of HERCEPTIN® that inhibit cell growth but allow cell survival, show an increase in the amount of MUC1 and especially MUC1 that is expressed. This increase in the amount of MUC1 or MUC1* that is expressed significantly raises the amount of HERCEPTIN® that is required to inhibit cell growth by 50% (IC50) and eventually causes complete resistance to HERCEPTIN®. This scenario of drug resistance is not limited to HERCEPTIN®. Elevated or increased expression of MUC1 predicts that the patient is at risk for acquiring early drug resistance to a variety of cancer drugs, including cytotoxic drugs like taxol, doxorubicin and cyclophosphamide. Increased expression of MUC1 or MUC1 predicts that the patient will acquire early drug resistance to targeted therapeutics as well, especially therapeutics that target components of the MAP kinase signaling pathway.

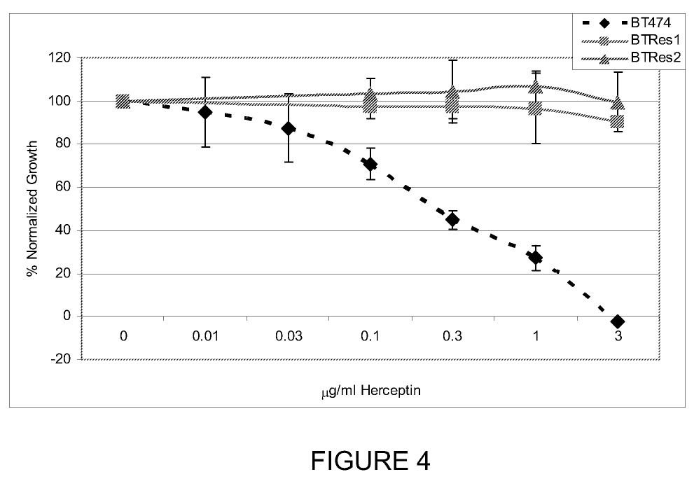

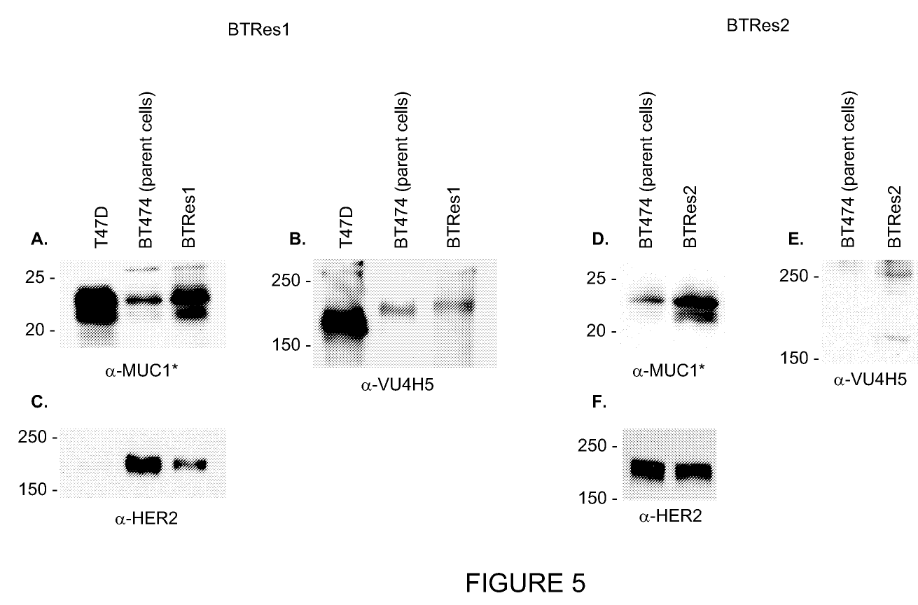

Breast cancer cell line BT474 expresses high levels of the HER2 receptor and low levels of MUC11. HER2 is a growth factor co-receptor that triggers cell growth when ligand binding allows HER2 to heterodimerize with one of the ErbB family of receptors. Treatment with HERCEPTIN® inhibits the growth of BT474 and other cells that overexpress HER2 by blocking its dimerization domain. We induced BT474 cells to acquire HERCEPTIN® resistance by culturing the cells in sub-lethal levels of HERCEPTIN® for prolonged periods of time. Two pools of BT474 cells, BTRes1 and BTRes2, were cultured according to standard protocols except that HERCEPTIN® was added to the cell growth media at a concentration of 1 ug/ml. After eight (8) weeks of culturing in sub-lethal levels of HERCEPTIN®, BTRes1 and BTRes2 were tested alongside the parent cells, BT474, to determine whether or not there had been a change in their response to HERCEPTIN®. FIG. 4 shows that treatment with HERCEPTIN® at concentrations that had previously caused 90-100% inhibition of cell growth, had no effect on the resistant cells. Because we had previously seen that the transfection of MUC1* into MUC1-negative cells rendered the cells resistant to cell death induced by standard chemo therapy agents, we wondered if the expression levels of MUC1* had changed as a result of acquiring resistance to HERCEPTIN®. Western blot analysis of the resistant cells (FIG. 5) showed a significant increase in the expression of MUC1*. HERCEPTIN® resistant cells expressed 4-6 times more MUC1* than the parent cell line, while the amount of full-length MUC1 appeared comparable to amounts present in the parent cells. These results are consistent with the idea that the expression of MUC1 had increased and that the rate of receptor cleavage had also increased so that what was left on the cell surface was the growth factor receptor form of MUC1, MUC1*. In one resistant pool, the expression of HER2 was relatively unchanged, while in the other pool, expression of HER2 was reduced by approximately 25%. These results confirm that acquired HERCEPTIN® resistance is accompanied by an increase in the amount of MUC1* that is expressed.

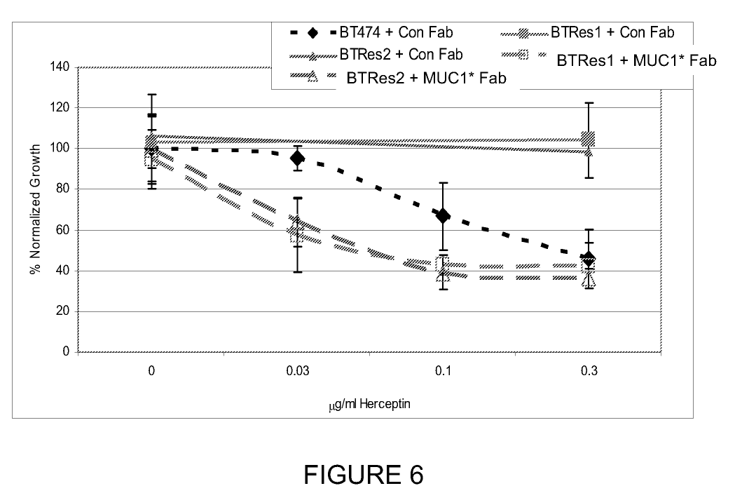

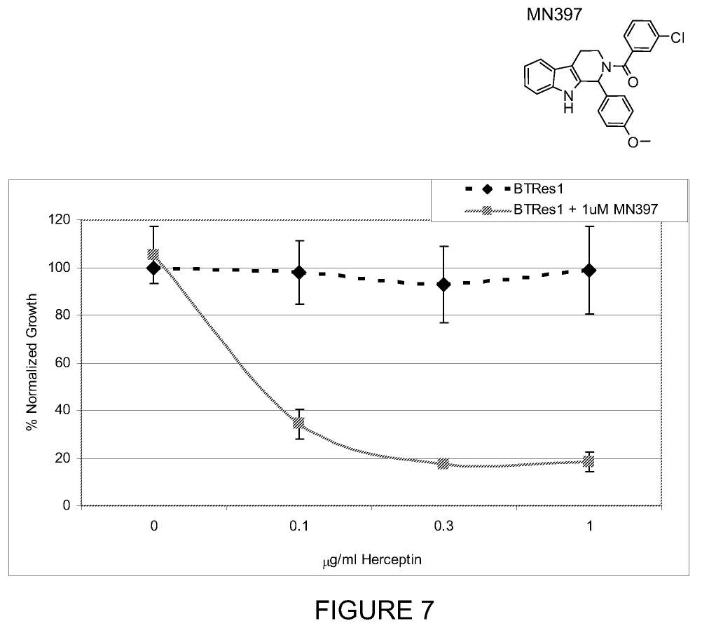

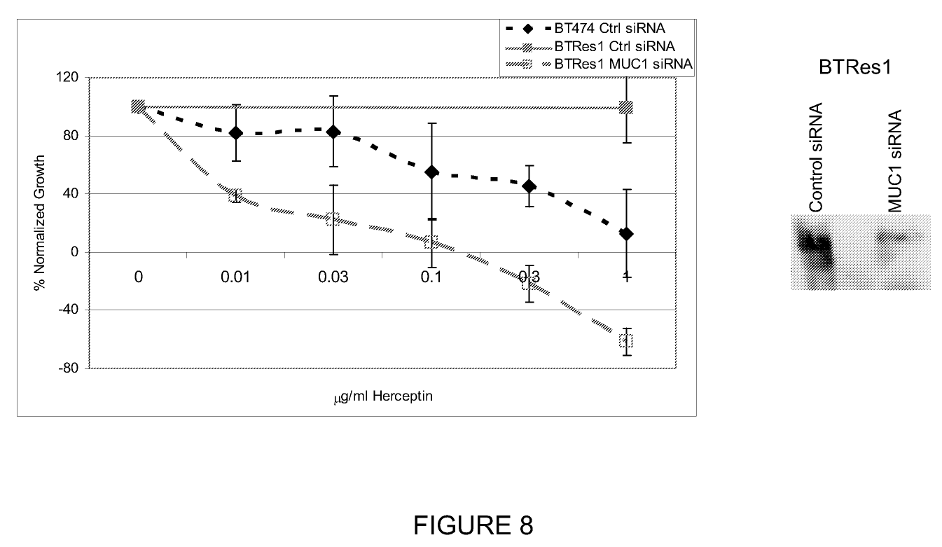

In our previous work, we had successfully inhibited the growth of breast cancer cell lines that inherently overexpressed MUC1* by blocking ligand-induced dimerization of MUC1*. Thus, we speculated that acquired HERCEPTIN® resistance might be reversed by inactivating MUC1*. Dimerization of MUC1* can be blocked by the monovalent Anti-MUC1 Fab or small molecules that bind to the extracellular domain of MUC1*. BTRes1 and BTRes2 HERCEPTIN® resistant cells were treated with 2.5 ug/ml of an anti-MUC1* Fab and variable concentrations of HERCEPTIN®. FIG. 6 shows that treating cells with the combination of a MUC1* disabling anti-MUC1* Fab and HERCEPTIN® restored the therapeutic effect of HERCEPTIN® and the effect was dose-dependent. Similarly, when the HERCEPTIN® resistant cells were treated with HERCEPTIN® and MN397, a small molecule that binds to the extracellular domain of MUC1*, the resistant cells became HERCEPTIN® sensitive (FIG. 7). To verify that the increase in MUC1* expression was in fact causing the drug resistance, we suppressed MUC1 expression using stably transfected MUC1-specific siRNA. FIG. 8 shows that suppression of MUC1 suppresses the acquired drug resistance.

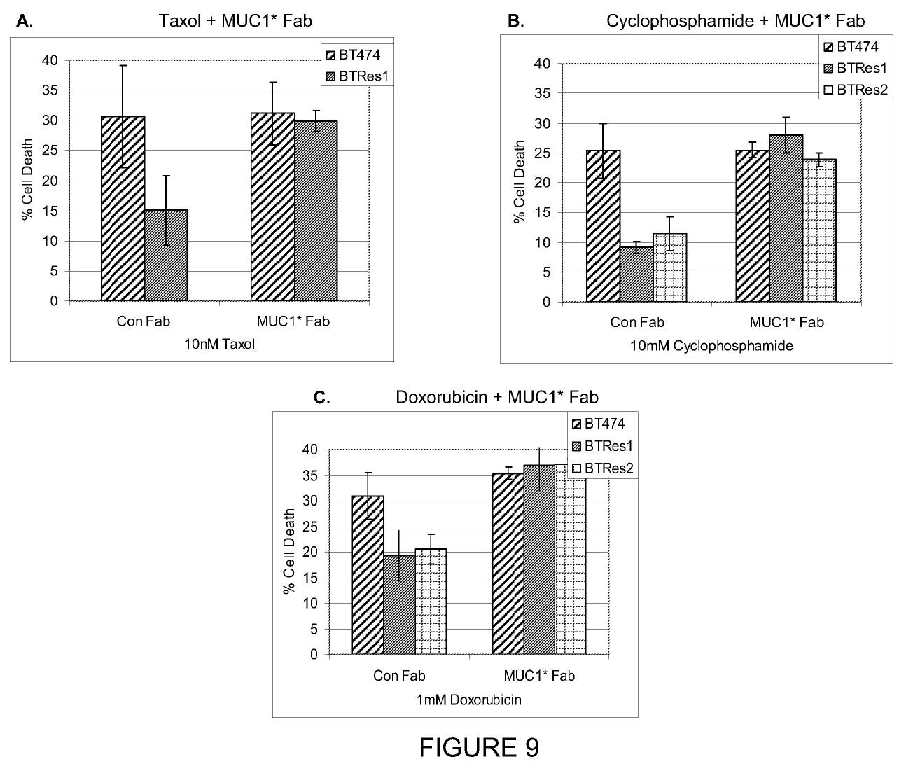

The cells that had acquired HERCEPTIN® resistance were tested to determine whether they also had acquired multi-drug resistance. Our tests showed that cells that overexpress MUC1* when they become resistant to a drug, like HERCEPTIN®, also acquire resistance to a broad spectrum of drugs. FIGS. 9A-C show that cells that had acquired resistance to HERCEPTIN®, also acquired resistance to the cytotoxic anti-cancer drugs, taxol, cyclophosphamide, and doxorubicin. Disabling MUC1*, by binding a monovalent agent to its extracellular domain, reversed the acquired multi-drug resistance in the absence of HERCEPTIN®, once again confirming the role of MUC1* in drug resistance.

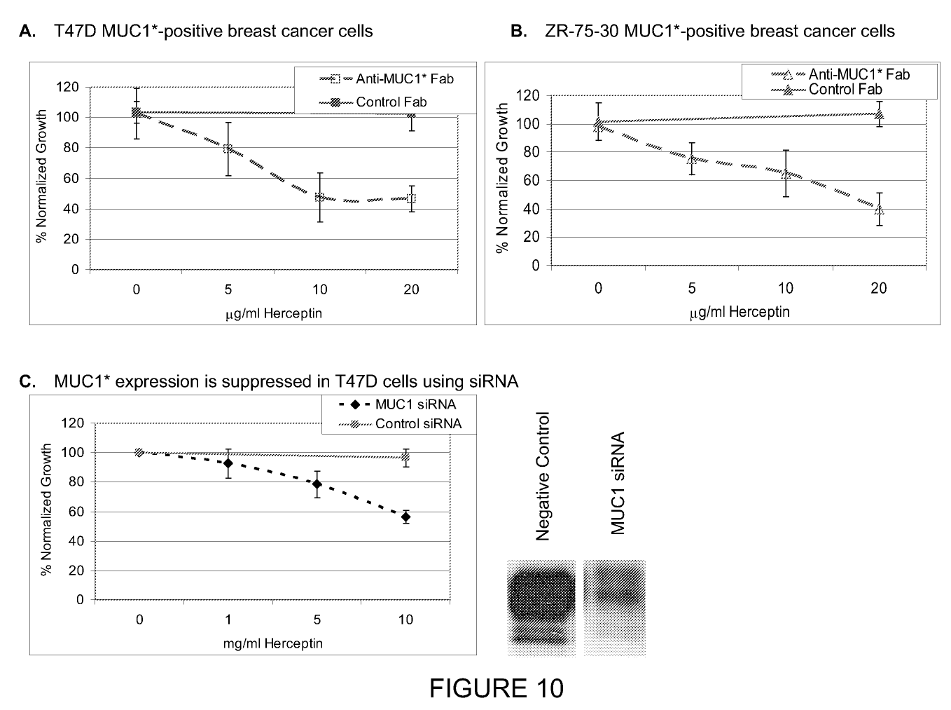

We also investigated whether cancer cells that intrinsically expressed high levels of MUC1* were resistant to certain drugs. We found that HERCEPTIN®had no therapeutic effect on several cancer cell lines that expressed high levels of MUC1*. However, treatment with a MUC1* disabling agent, such as a anti-MUC1* Fab, rendered the cells sensitive to HERCEPTIN-mediated growth inhibition (see FIG. 10). When MUC1* expression was suppressed using MUC1-specific siRNA, cells became HERCEPTIN® sensitive, which confirms that the previously observed drug resistance was due to the presence of high levels of MUC1* (see FIG. 10C). MUC1* may also be disabled by binding agents to its intracellular tail such that interactions with signaling proteins are clocked.

Therefore, the presence of even low levels of MUC1* on a patient's tumor specimen is an indicator that the patient is at risk for acquiring drug resistance. Tumors that are characterized by medium or high levels of MUC1* are at an even greater risk of acquiring early drug resistance. These patients should be treated with anti-cancer agents that disable MUC1* and/or the MAP kinase signaling pathway. Prior to cancer treatment, patients whose cancers show a medium to high level of MUC1 or MUC1* should be treated with combined therapies that include drugs that inhibit cell growth mediated by the MAP kinase signaling pathway. In a preferred embodiment, breast cancer patients whose tumors are characterized by HER2 overexpression and are MUC1 or MUC1* positive should be treated with HERCEPTIN®, or other agent that inhibits signaling through the HER2 receptor, in combination with drugs that inhibit MUC1* and/or the MAP kinase signaling pathway.

Thus, determining a level of MUC1 or MUC1* is a diagnostic or prognostic indicator of a patient's susceptibility to acquiring early drug resistance in the treatment of a cancer. Because MUC1* is a growth factor receptor that drives the growth of cancer cells and cancer stem cells, an increased expression of MUC1* is also a prognostic indicator of cancer recurrence and life expectancy. A preferred method of determining whether or not a tumor specimen expresses an amount of MUC1* that would have prognostic value is determined as follows.

Biopsy specimens are stained according to standard immunohistochemistry (IHC) methods using an antibody that recognizes the MGFR portion of MUC1 (also referred to here as anti-MUC1*). A pathologist, or a skilled technician by preferably using an automated instrument, rates the amount of membrane staining according to 0-3 plus signs (0, +, ++, +++). A cancer is determined to be MUC1* negative if there is no (0) or weak (+) MUC1* membrane staining on less than 10% of the tissue specimen. The tissue specimen is MUC1*-positive if there is MUC1* membrane staining with an intensity of 2 (++) over more than 10% of the tissue specimen. The risk of acquiring drug resistance or cancer recurrence or death is only moderately increased with ++ MUC1*-staining over less than 30% of the tumor specimen. The risk is significant with ++ MUC1*-staining over more than 30% of the tumor. The risk is greatest with an intensity IHC MUC1*-staining of +++ over more than 30% of the tumor and predicts cancer recurrence, metastasis and shortened life expectancy. These patients should be treated aggressively and with a therapy that includes a MUC1* disabling agent.

In a less preferred method, risk assessment can also be determined by measuring an amount of MUC1 using an antibody such as VU4H5 that binds to epitopes in the distal region of the molecule. Because this portion of the receptor is frequently shed from the cell surface, staining using these antibodies would not be limited to membrane staining. Fluorescent in situ hybridization (FISH) as well as other methods that quantify the amount of the MUC1 gene (nucleic acid-based) that cells express are also used to determine levels of MUC1 expression that are predictive of acquisition of drug resistance, metastasis, recurrence and life expectancy. In this aspect of the invention the level of MUC1 encoding nucleic acid is compared to the level of cep-17 (or other normally expressed gene) encoding nucleic acid. A ratio of about 1.8 or higher for MUC1:CEP17 is considered a MUC1-positive cancer. The risks associated with MUC1 aberrant expression increase as the ratio of MUC1: CEP17 increase, where a ratio of 2.2 is high risk and over 2.5 is very high risk and over 3.0 is extremely high risk.

Other techniques can be employed to determine whether the amount of MUC1 or MUC1 is aberrantly expressed. Cancer cells extracted from a patient are lysed and analyzed using MUC1 or MUC1*-specific antibodies and sandwich assay techniques such as ELISA or bead assays and the like. MUC1 or MUC1 would be determined to be overexpressed if their expression was more than 2-3 standard deviations above non-cancerous cells of similar type taken from a healthy population. Similarly, one can indirectly assess the amount of MUC1 that a patient's cancer expresses by measuring the amount of NM23 that the patient expresses, as we have determined that NM23 is the activating ligand of MUC11. An additional method of tracking MUC1 activation, and responsiveness of cancers to MUC1*-targeted treatment is to develop antibodies that detect its ligand, NM23-H1. This antibody is used in a sandwich ELISA test or other antibody detection tests in the blood, lymph, or space around biopsied tissues. Also, antibody detection (biopsies, Western or dot blot, intracellular FACS) of MMP-14 or TACE, the cleavage enzymes of MUC1, which result in generation of MUC1 on the surface of cancer cells may also be an indication of a patient's susceptibility to acquiring early drug resistance in the treatment of a cancer. Measuring an amount of MMP14 or ADAM-17, which are enzymes that cleave MUC1 to the growth factor receptor form MUC1 is another way of assessing how much MUC1a patient's cancer expresses and also for determining a patient's response to MUC1-targeting drugs. The amount of MUC1 that is present on the surface of the tumor cells can also be determined indirectly by measuring an amount of shed MUC1 in a patient's blood or other bodily fluid. On cancer cells, MUC1 is cleaved and the bulk of the extracellular domain is “shed” or released from the cell surface into the circulation. Detecting an amount of this shed portion of MUC1 that is devoid of the first membrane proximal 35-45 amino acids of the extracellular domain is an indirect measure of the amount of MUC1* that is in the tumor.

Therapies that combine HER2-targeting agents and MUC1-targeting agents are preferred because the two agents act synergistically and combined treatment reduces the risk of developing resistance to HERCEPTIN® by overexpression of MUC1. Therapies that combine HER2-targeting agents and MUC1*-targeting agents are especially preferred. Therapies that combine HER2-targeting agents, MUC1*-targeting agents and cytotoxic anti-cancer agents, such as Cisplatin, AraC, Etoposide, cyclophosphamide, taxol, and doxirubicin are still more preferred.

Because an increase in the expression of MUC1 accompanies the acquisition of drug resistance, and increases the risk of metastasis and poor prognosis, the present invention provides a method of monitoring a patient receiving cancer drug treatment in which levels of MUC1, MUC1* or MUC1-associated factors that the patient produces are periodically measured and wherein an increase in these levels indicates to the physician that changes in the patient's therapy need to be made. Such changes in treatment include more aggressive treatment and the inclusion of therapies that are designed to suppress or disable MUC1. Especially preferred are therapies designed to disable MUC1*, including treatment with, monovalent anti-MUC1* antibodies, such as Fabs and single chain ScFv antibodies as well as chemical compositions that bind to the extracellular domain of MUC1*. MUC1* disabling agents that can be used as anti-cancer therapeutics include but are not limited to monovalent anti-MUC1* Fabs, chemical agents that bind to the PSMGFR region of the MUC1 protein, bispecific antibodies wherein at least one of the recognition regions binds to the PSMGFR region of MUC1, RNAi that suppresses expression of MUC1, MMP-14, ADAM-17 (TACE) and/or NM23, and agents that disrupt the dimerization domain of NM23. Bispecific antibodies may include antibodies that bind to the extracellular domain of MUC1* with one domain and another tumor-associated antigen with the other recognition domain. Suitable tumor-associated antigens include the extracellular domain of HER2 and other ErbB family members.

Antibodies

Peptides used for antibody production may or may not be glycosylated prior to immunizing animals. The sequence of these peptides need not exactly reflect the sequence of MUC1 receptor as it exists in the general population. For example, the inventors observed that antibodies raised against the PSMGFR peptide variant var-PSMGFR (SEQ ID NO:12), having an “-SPY-” motif have a higher affinity and greater specificity for the MUC1 protein than antibodies raised against the actual native sequence (i.e. nat-PSMGFR, SEQ ID NO:10), having an “-SRY-” motif. One may also, in certain embodiments, introduce mutations into the PSMGFR peptide sequence to produce a more rigid peptide that may enhance antibody production. For example the R to P mutation in the var-PFMGFR sequence of SEQ ID NO:12 may actually have provided a more rigid peptide and was thus more immunogenic. Another method for producing antibodies against regions of peptides that are not particularly immunogenic, such as the IBR or TPSIBR is to tag the specific peptide sequence with an irrelevant sequence in which the amino acids are of the D-form and thus act to stimulate the immune response of the host animal. Peptide sequences that are used to immunize animals for antibody production may also be glycosylated. The MUC1 peptide sequences that were used herein for drug screening and to generate cognate antibodies were derived from the human species of MUC1. Since there is considerable conservation across species for the PSMGFR and IBR and some portions of the UR, it is anticipated that MUC1 peptides whose sequences are derived from other species can also be used in drug screens and to generate antibodies for these same purposes.

In certain aspects, the invention provides antibodies or antigen-binding fragments thereof. In one embodiment, the invention provides an antibody or antigen-binding fragment that specifically binds to MGFR. In certain embodiments, the above-mentioned antibodies or antigen-binding fragments thereof specifically bind to PSMGFR. In certain such embodiments, the antibodies or antigen-binding fragments thereof can specifically bind to the amino acid sequence set forth in SEQ ID NO:10 or a functional variant or fragment thereof comprising up to 15 amino acid additions or deletions at its N-terminus or comprising up to 20 amino acid substitutions; in other embodiments, it specifically binds to the amino acids set forth in SEQ ID NO:10 or a functional variant or fragment thereof comprising up to 10 amino acid substitutions; in other embodiments, the antibodies or antigen-binding fragments thereof specifically bind to the amino acid set forth in SEQ ID NO:10 or a functional variant or fragment thereof comprising up to 5 amino acid substitutions; and in yet another embodiments the antibodies or antigen-binding fragments thereof specifically bind to the amino acid sequence set forth in SEQ ID NO:10. In certain embodiments, the antibody or antigen-binding fragment of the invention is a human, humanized, xenogenic or a chimeric human-non-human antibody or antigen-binding fragment thereof. In certain embodiments, the antibodies or antigen-binding fragments thereof of the invention comprise an intact antibody or an intact single-chain antibody. For antibodies or antigen-binding fragments that are monovalent, in certain embodiments, they may comprise a single-chain Fv fragment, a Fab′ fragment, a Fab fragment, or a Fd fragment. For antibodies or antigen-binding fragments of the invention that are bivalent, certain embodiments comprise an antigen-binding fragment that is a F(ab′)₂. In certain such compositions, the antibody or antigen-binding fragment thereof can be polyclonal, while in other embodiments it can be monoclonal.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

As is now well known in the art, the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205, which are incorporated by reference herein in their entirety. Such antibodies, or fragments thereof are within the scope of the present invention.

In certain embodiments, fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In certain embodiments the present invention comprises methods for producing the inventive antibodies, or antigen-binding fragments thereof, that include any one of the step(s) of producing a chimeric antibody, humanized antibody, single-chain antibody, Fab-fragment, F(ab′)₂ fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor, 1988. The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogeneic antibodies. The general principle for the production of xenogeneic antibodies such as human antibodies in mice is described in, e.g., WO 91/10741, WO 94/02602, WO 96/34096 and WO 96/33735. As discussed below, the antibodies, of the invention may exist in a variety of forms (besides intact antibodies; including, for example, antigen binding fragments thereof, such as Fv, Fab and F(ab′)2, as well as in single chains (i.e. as single chain antibodies); see e.g., WO88/09344.

Thus, as will be apparent to one of ordinary skill in the art, the present invention also provides, in certain embodiments, for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Chemical Derivatives of Antibodies and Formulations

In certain embodiments, the present invention relates to compositions comprising the aforementioned antibodies or antigen-binding fragments of the invention or chemical derivatives thereof. The composition of the present invention may further comprise a pharmaceutically acceptable carrier. The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes, e.g., for intranasal administration. Formulations for rectal or vaginal administration may be presented as a suppository with a suitable carrier.

A therapeutically effective dose refers to that amount of antibodies and/or antigen-binding fragments of the invention ameliorate the symptoms or conditions of the disease being treated. Therapeutic efficacy and toxicity of such compositions can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The biological activity of the antibodies and/or antigen binding fragments thereof, of the invention indicates that they may have sufficient affinity to make them candidates for drug localization to cells expressing the appropriate surface structures, e.g. MGFR. Thus, targeting and binding to cells of the antibodies and/or antigen binding fragments thereof, of the invention could be useful for the delivery of therapeutically or diagnostically active agents (including targeting drugs, DNA sequences, RNA sequences, lipids, proteins and gene therapy/gene delivery. Thus, the antibody and/or antigen binding fragments thereof, of the invention can be labeled (e.g., fluorescent, radioactive, enzyme, nuclear magnetic, colloid, other signaling entity, etc.) and used to detect specific targets in vivo or in vitro including “immunochemistry” like assays in vitro. In vivo they could be used in a manner similar to nuclear medicine imaging techniques to detect tissues, cells, or other material expressing MGFR. Another method of the invention involves using antibodies that bind to the MGFR portion of the MUC1 receptor as a method for sorting and/or isolating cells that need to be expanded. Once sorted, these cells would be expanded in vitro. New genetic material, for example that codes for co-receptors and/or activating ligands, may be added to these selected cells either before or after expansion. Activating antibodies may be depleted from the cell population before introduction to the subject. Yet another method involves delivering a therapeutically active agent to a patient. The method includes administering at least one antibody or an antigen-binding fragment thereof and the therapeutically active agent to a patient. Preferably, the therapeutically active agent is selected from drugs, DNA sequences, RNA sequences, proteins, lipids, and combinations thereof.

Administration and Dosage

When used therapeutically, the agents of the invention are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.01 mg/kg to about 1000 mg/kg. It is expected that dose ranging from 1-500 mg/kg, and preferably doses ranging from 1-50 mg/kg will be suitable. In other embodiments, the agents will be administered in doses ranging from 1 μg/kg/day to 10 mg/kg/day, with even more preferred doses ranging from 1-200 μg/kg/day, 1-100 μg/kg/day, 1-50 μg/kg/day or from 1-25 μg/kg/day. In other embodiments, dosages may range from about 0.1 mg/kg to about 200 mg/kg, and most preferably from about 0.2 mg/kg to about 20 mg/kg. These dosages can be applied in one or more dose administrations daily, for one or more days.

The agent of the invention should be administered for a length of time sufficient to provide either or both therapeutic and prophylactic benefit to the subject. Generally, the agent is administered for at least one day. In some instances, the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a event associated with tumor or cancer, provided still that such doses achieve the medically desirable result. On the other hand, it may be desirable to administer lower doses in order to maintain the medically desirable result once it is achieved. In still other embodiments, the same dose of agent may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time there between as if such time was explicitly recited herein.

In one embodiment, daily doses of active agents will be from about 0.01 milligrams/kg per day to 1000 milligrams/kg per day. It is expected that oral doses in the range of 50 to 500 milligrams/kg, in one or several administrations per day, will yield the desired results. Dosage may be adjusted appropriately to achieve desired levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of agents.

Preferably, such agents are used in a dose, formulation and administration schedule which favor the activity of the agent and do not impact significantly, if at all, on normal cellular functions.

In one embodiment, the degree of activity of the agent is at least 10%. In other embodiments, the degree of activity of the drug is as least 20%, at least 30%, at least 40%, at least 50%, at least 60%, at least 70%, at least 80%, at least 90%, or at least 95%.

When administered to subjects for therapeutic purposes, the formulations of the invention are applied in pharmaceutically acceptable amounts and in pharmaceutically acceptable compositions. Such a pharmaceutical composition may include the agents of the invention in combination with any standard physiologically and/or pharmaceutically acceptable carriers which are known in the art. The compositions should be sterile and contain a therapeutically effective amount of the agent in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V)

Suitable preservatives include benzalkonium chloride (0.003-0.03% W/V); chlorobutanol (0.3-0.9% W/V); parabens (0.01-0.25% W/V) and thimerosal (0.004-0.02% W/V)

A variety of administration routes are available. The particular mode selected will depend, of course, upon the particular combination of drugs selected, the severity of the disease condition being treated, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, other mucosal forms, direct injection, transdermal, sublingual or other routes. “Parenteral” routes include subcutaneous, intravenous, intramuscular, or infusion. Direct injection may be preferred for local delivery to the site of the cancer. Oral administration may be preferred for prophylactic treatment e.g., in a subject at risk of developing a cancer, because of the convenience to the patient as well as the dosing schedule.

Chemical/physical vectors may be used to deliver the agents of the invention to a target (e.g. cell) and facilitate uptake thereby. As used herein, a “chemical/physical vector” refers to a natural or synthetic molecule, other than those derived from bacteriological or viral sources, capable of delivering the agent of the invention to a target (e.g. cell).

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., v. 6, p. 77 (1981)). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular (e.g. tissue), such as (e.g. the vascular cell wall), by coupling the liposome to a specific ligand such as a monoclonal antibody, sugar, glycolipid, or protein.

Liposomes are commercially available from Gibco BRL, for example, as LIPOFECTIN™. and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, V. 3, p. 235-241 (1985).

In one particular embodiment, the preferred vehicle is a biocompatible micro particle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the agent of the invention is encapsulated or dispersed within the biocompatible, preferably biodegradable polymeric matrix disclosed in PCT/US/03307. The polymeric matrix preferably is in the form of a micro particle such as a micro sphere (wherein the agent is dispersed throughout a solid polymeric matrix) or a microcapsule (wherein the agent is stored in the core of a polymeric shell). Other forms of the polymeric matrix for containing the agents of the invention include films, coatings, gels, implants, and stents. The size and composition of the polymeric matrix device is selected to result in favorable release kinetics in the tissue into which the matrix device is implanted. The size of the polymeric matrix devise further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the devise is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described agents for use as a medicament, methods for preparing the medicament and methods for the sustained release of the medicament in vivo.

The compositions may conveniently be presented in unit dosage form and may be prepared by any of the methods well known in the art of pharmacy. All methods include the step of bringing the therapeutic agents into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion

Other delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; liposomes; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation

Use of a long-term sustained release implant may be particularly suitable for treatment of established disease conditions as well as subjects at risk of developing the disease. “Long-term” release, as used herein, means that the implant is constructed and arranged to deliver therapeutic levels of the active ingredient for at least 7 days, and preferably 30-60 days. The implant may be positioned at a site of injury or the location in which tissue or cellular regeneration is desired. Long-term sustained release implants are well known to those of ordinary skill in the art and include some of the release systems described above

The therapeutic agent may be administered in alone or in combination with other agents including proteins, receptors, co-receptors and/or genetic material designed to introduce into, upregulate or down regulate these genes in the area or in the cells. If the therapeutic agent is administered in combination the other agents may be administered by the same method, e.g. intravenous, oral, etc. or may be administered separately by different modes, e.g. therapeutic agent administered orally, administered intravenously, etc. In one embodiment of the invention the therapeutic agent and other agents are co-administered intravenously. In another embodiment the therapeutic agent and other agents are administered separately

Other agents that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Antibody Production

Antibodies that bind to the MGFR portion of the MUC1 receptor, referred to herein as anti-PSMGFR are described in detail in PCT Application No. PCT/US2004/027954 (WO 2005/019269), in particular in Example 8 of the PCT Application. Antibody production is also described in PCT Application No. PCT/US2005/032821, in particular in Example 2 of the PCT Application. Inventive antibodies were raised against the PSMGFR portion of the MUC1 receptor, in particular nat-PSMGFR or var-PSMGFR shown in Table 1 using standard methods of antibody production. Rabbit polyclonal antibodies were produced and purified by column chromatography in which the immunizing peptide was attached to the chromatography column beads. The antibodies, anti-nat-PSMGFR and anti-var-PSMGFR, were shown to specifically and sensitively bind to the MGFR portion of the MUC1 receptor.

### Example 2

### Preparation of Tissue Specimens

Tissue specimens pictured in FIGS. 7-15 were prepared using methods previously described in PCT Application No. PCT/US2005/032821, in particular in Example 3 of the PCT Application. Formalin fixed, paraffin embedded tissue specimens were tested for reactivity to two antibodies that recognize different epitopes on the MUC1 receptor: 1) a rabbit polyclonal antibody, anti-PSMGFR, that binds to the PSMGFR portion of the MUC1 receptor that remains attached to the cell surface after receptor shedding; and 2) a commercially available mouse monoclonal, VU4H5 (Santa Cruz, Calif.) that binds to a sequence in the tandem repeat section of the receptor. One section from each block was stained with hemotoxin and eosin (H&E) to aid in assessing tumor grade. A table summarizing the results of larger group of tissue specimens including pathologist's score for each is shown as FIG. 18. MUC1* staining refers to staining with rabbit polyclonal anti-MUC1* and MUC1 staining refers to staining with VU4H5.

### Example 3

### Making HERCEPTIN® Resistant Cells

Two pools of the breast tumor line BT474 (ATCC) were made resistant by culturing in RPMI medium containing HERCEPTIN® at a final concentration of 1 μg/ml for 8 weeks. HERCEPTIN® resistance was then verified. Briefly, BT474 cells, and resistant cells (BTRes1 and BTRes2) were plated in 96 well plates at 10,000 cells/well, six wells per condition. The following day, zero hour counts were taken, and medium was changed in the remaining wells to RPMI containing HERCEPTIN® to final concentrations of 0, 0.01, 0.03, 0.1, 0.3, 1, and 3 ug/ml. Three days later, the remaining cells were counted using a hemocytometer. BTRes 1 and BTRes2 cells showed no effects of HERCEPTIN® at these concentrations, while BT474 cells reached 50% growth inhibition at a HERCEPTIN® concentration between 0.1 and 0.3 μg/ml.

### Example 4

### Anti-MUC1* Fab Gragment Generation

A rabbit polyclonal antibody generated by standard methods using the MUC1 derived peptide var-PSMGFR (SEQ ID NO:12), for immunization. To prepare a monovalent Fab fragment from the MUC1* antibody, a papain digestion method was employed (Pierce). The antibody was effectively digested with papain, then purified by Protein A-agarose affinity column chromatography. Those Fab fragments were tested along with its parental antibody in competition ELISA to make sure the Fab fragment is capable of binding to its antigen, the MUC1* peptide. As shown in FIG. 6, Fab fragments were competing with its parent antibody effectively. Fab fragments were then tested in cell-based inhibition assay, MUC1-positive cell growth was inhibited but the growth of MUC 1-negative cells was not.

### Example 5

### Reversing Acquired Drug Resistance by Disabling Muc1*

Reversal of acquired HERCEPTIN® resistance was demonstrated using a MUC1-1-disabling Fab. BT474, BTRes1 and BTRes2 Cells were plated in 96 well plates at 10,000 cells/well, six wells/condition. The following day, zero hour counts were taken, and medium was changed in the remaining wells to RPMI containing HERCEPTIN® to final concentrations of 0, 0.03, 0.1, and 0.3 ug/ml, in the presence of 2.5 ug/ml Anti-MUC1* Fab (Minerva Biotechnologies, Mahanta, et al, 2008; added to BTRes1 and BTRes2), or 2.5 ug/ml Control Fab (Jackson Immunoresearch 315-007-008; added to BT474, BTRes1, and BTRes2). One set of wells was left untreated. Three days later, cells were counted, and Percent Normalized Growth was calculated. Addition of the MUC1*-specific Fab restored HERCEPTIN® sensitivity to BTRes1 and BTRes2, resulting in a 50% growth inhibition concentration between 0.03 and 0.1 μg/ml.

### Example 6

### MUC1-Specific RNAi is Used Therapeutically to Block MUC1*-Mediated Cancer Cell Growth and to Reverse Acquired Drug Resistance

BT474 cells were transfected in triplicate with control siRNA (Santa Cruz Biotechnologies; sc-37007), and BTRes1 cells were transfected in triplicate with control or MUC1-specific siRNA (Santa Cruz Biotechnologies sc-35985). One microliter of 10 μM siRNA was added to 100 ul of OptiMEM medium (Invitrogen 22600134). Six microliters of HiPerfect reagent (Qiagen 301704) were then added to each tube. After vortexing, tubes were incubated at room temperature for 20 minutes. Meanwhile, cells were trypsinized, pelleted and resuspended in fresh RPMI without HERCEPTIN®. 5×10⁵ cells in 2.3 ml RPMI were combined with the OptiMEM with siRNA/HiPerfect complexes and added to a well of a 6 well plate. Two days later, cells were re-transfected by the same method, and plated in 96 well plates, at 10,000 cells per well, five wells per condition. Leftover cells were plated in six well plates. The following day, zero hour counts were taken, and medium was changed in the remaining wells to RPMI containing HERCEPTIN® to final concentrations of 0, 0.01, 0.03, 0.1, 0.3, and 1 ug/ml. The following day, cells plated in 6 well plates were harvested and pelleted for Western analysis to evaluate siRNA efficacy. Three days later, cells were counted, and Percent Normalized Growth was calculated. Downregulation of MUC1* protein by transfection of MUC1-specific siRNA into BTRes1 cells, resulted in restoration of HERCEPTIN® sensitivity, resulting in a 50% growth inhibition concentration of less than 0.01 ug/ml.

### Example 7

### Human Cancer Stem Cells Express More MUC1* than the Pool of Cancer Cells from which they were Isolated

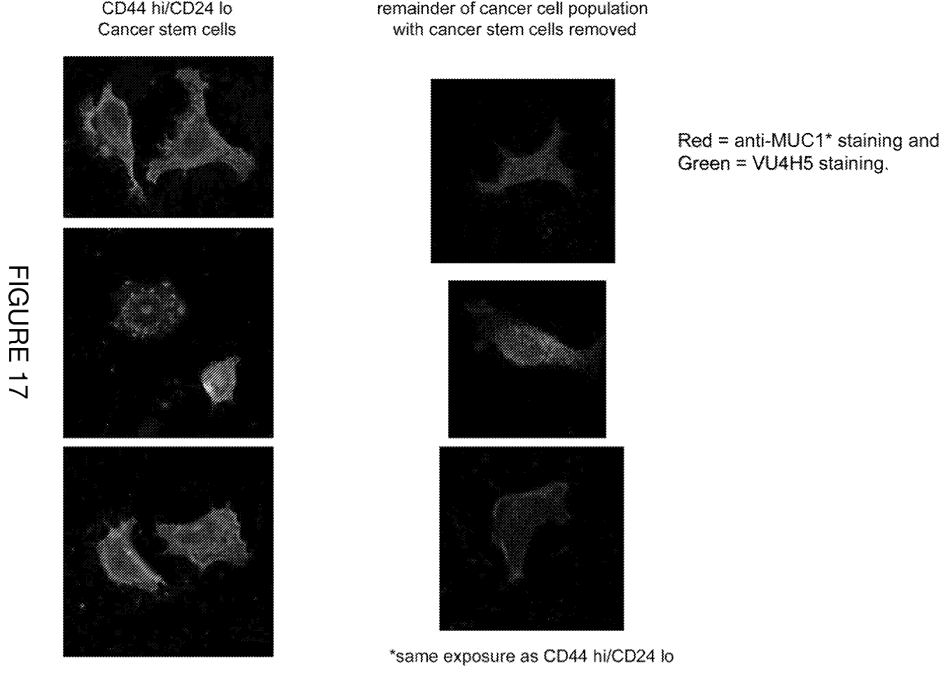

### See FIG. 17

Cancer stem cells, defined as CD44hi/CD24low were separated away from the mixed population of human breast cancer MCF-7 cells (ATCC HTB-22) using FACS. Cells from the mixed pool and cells from the cancer stem cell pool were prepared for antibody staining and subsequent imaging as follows. 50,000 cells were plated into each of 8 wells of an 8 well chamber slide. The following day, cells were fixed in 4% paraformaldehyde in Sodium Cacodylate buffer (pH 7.4), washed in PBS, and blocked in 2.5% donkey serum and 2.5% BSA in PBS. Cells were stained with anti-MUC1* rabbit polyclonal antibody (Minerva Biotechnologies, 1:250 dilution of 0.77 mg/ml stock), and anti-MUC1 Tandem Repeat antibody (VU4H5 Santa Cruz Biotechnologies, 1:50 dilution of 0.2 mg/ml stock). Cells were washed, and primary antibodies were detected using an anti-rabbit Alexa-555 antibody (Invitrogen, 1:200 dilution of a 2 mg/ml stock), and anti-mouse Alexa-488 antibody (Invitrogen, 1:200 dilution of a 2 mg/ml stock). Cells were washed, and cover glass was mounted using an anti-fade mounting medium (Biomeda), and sealed with clear nail polish. Cells were visualized and images were acquired by confocal microscopy. FIG. 17 shows that the cancer stem cells (CD44hi/CD24lo) have much more MUC1* and MUC1 than the remaining pool from which they were selected. Red fluorescence is anti-MUC1*. Green fluorescence is VU4H5.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

TABLE 1

Peptide sequences (listed from N-terminus
to C-terminus):

Full-length MUC1 Receptor (Mucin 1 precursor,
Genbank Accession number: P15941)

(SEQ ID NO:1)

MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA ASANL

N-terminal MUC-1 signaling sequence for directing MUC1 receptor and truncated isoforms to cell membrane surface. Up to 3 amino acid residues may be absent at C-terminal end as indicated by variants in SEQ ID NOS:2, 3 and 4.

| | |
|---|---|
| MTPGTQSPFFLLLLLTVLT. | (SEQ ID NO:2) |
| MTPGTQSPFFLLLLLTVLT VVTA | (SEQ ID NO:3) |
| MTPGTQSPFFLLLLLTVLT VVTG | (SEQ ID NO:4) |

A truncated MUC1 receptor isoform having nat-PSMGFR at its N-terminus and including the transmembrane and cytoplasmic sequences of a full-length MUC1 receptor (“nat-PSMGFRTC isoform”—An example of “PSMGFRTC”—shown excluding optional N-terminus signal sequence, which may be cleaved after translation and prior to expression of the receptor on the cell surface):

(SEQ ID NO:5)

G TINVHDVETQ FNQYKTEAAS RYNLTISDVS VSDVPFPFSA
QSGAGVPGWG IALLVLVCVL VALAIVYLIA LAVCQCRRKN
YGQLDIFPAR DTYHPMSEYP TYHTHGRYVP PSSTDRSPYE
KVSAGNGGSS LSYTNPAVAA ASANL

A truncated MUC1 receptor isoform having nat-PSMGFR and PSIBR at its N-terminus and including the transmembrane and cytoplasmic sequences of a full-length MUC1 receptor (“CM isoform”—shown excluding optional N-terminus signal sequence, which may be cleaved after translation and prior to expression of the receptor on the cell surface):

(SEQ ID NO:6)

GFLGLS NIKFRPGSVV VQLTLAFREG TINVHDVETQ FNQYKTEAAS
RYNLTISDVS VSDVPFPFSA QSGAGVPGWG IALLVLVCVL
VALAIVYLIA LAVCQCRRKN YGQLDIFPAR DTYHPMSEYP
TYHTHGRYVP PSSTDRSPYE KVSAGNGGSS LSYTNPAVAA
ASANL

A truncated MUC1 receptor isoform having nat-PSMGFR+PSIBR+Unique Region at its N-terminus and including the transmembrane and cytoplasmic sequences of a full-length MUC1 receptor (“UR isoform”—shown excluding optional N-terminus signal sequences):

(SEQ ID NO:7)

ATTTPASKST PFSIPSHHSD TPTTLASHST KTDASSTHHS
TVPPLTSSNH STSPQLSTGV SFFFLSFHIS NLQFNSSLED
PSTDYYQELQ RDISEMFLQI YKQGGFLGLS NIKFRPGSVV
VQLTLAFREG TINVHDVETQ FNQYKTEAAS RYNLTISDVS
VSDVPFPFSA QSGAGVPGWG IALLVLVCVL VALAIVYLIA
LAVCQCRRKN YGQLDIFPAR DTYHPMSEYP TYHTHGRYVP
PSSTDRSPYE KVSAGNGGSS LSYTNPAVAA ASANL

A truncated MUC1 receptor isoform including the transmembrane and cytoplasmic sequences of a full-length MUC1 receptor (“Y isoform”—shown excluding optional N-terminus signal sequence, which may be cleaved after translation and prior to expression of the receptor on the cell surface):

(SEQ ID NO:8)

GSGHASSTPG GEKETSATQR SSVPSSTEKN AFNSSLEDPS
TDYYQELQRD ISEMFLQIYK QGGFLGLSNI KFRPGSVVVQ
LTLAFREGTI NVHDMETQFN QYKTEAASRY NLTISDVSVS
DVPFPFSAQS GAGVPGWGIA LLVLVCVLVA LAIVYLIALA
VCQCRRKNYG QLDIFPARDT YHPMSEYPTY HTHGRYVPPS
STDRSPYEKV SAGNGGSSLS YTNPAVAATS ANL

A truncated MUC1 receptor isoform having nat-PSMGFR+PSIBR+Unique Region+Repeats at its N-terminus and including the transmembrane and cytoplasmic sequences of a full-length MUC1 receptor (“Rep isoform”—shown excluding optional N-terminus signal sequence, which may be cleaved after translation and prior to expression of the receptor on the cell surface):

(SEQ ID NO:9)

LDPRVRTSAP DTRPAPGSTA PQAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DNRPALGSTA
PPVHNVTSAS GSASGSASTL VHNGTSARAT TTPASKSTPF
SIPSHHSDTP TTLASHSTKT DASSTHHSSV PPLTSSNHST
SPQLSTGVSF FFLSFHISNL QFNSSLEDPS TDYYQELQRD
ISEMFLQIYK QGGFLGLSNI KFRPGSVVVQ LTLAFREGTI
NVHDVETQFN QYKTEAASRY NLTISDVSVS DVPFPFSAQS
GAGVPGWGIA LLVLVCVLVA LAIVYLIALA VCQCRRKNYG
QLDIFPARDT YHPMSEYPTY HTHGRYVPPS STDRSPYEKV
SAGNGGSSLS YTNPAVAAAS ANL

Native Primary Sequence of the MUC1 Growth Factor Receptor (nat-PSMGFR—an example of “PSMGFR”):

(SEQ ID NO:10)

GTINVHDVETQFNQYKTEAASRYNLTISDVSVSDVPFPFSAQSGA

Native Primary Sequence of the MUC1 Growth Factor Receptor (nat-PSMGFR—An example of “PSMGFR”), having a single amino acid deletion at the N-terminus of SEQ ID NO:10):

(SEQ ID NO:11)

TINVHDVETQFNQYKTEAASRYNLTISDVSVSDVPFPFSAQSGA

“SPY” functional variant of the native Primary Sequence of the MUC1 Growth Factor Receptor having enhanced stability (var-PSMGFR—An example of “PSMGFR”):

(SEQ ID NO:12)

GTINVHDVETQFNQYKTEAASPYNLTISDVSVSDVPFPFSAQSGA

“SPY” functional variant of the native Primary Sequence of the MUC1 Growth Factor Receptor having enhanced stability (var-PSMGFR—An example of “PSMGFR”), having a single amino acid deletion at the C-terminus of SEQ ID NO:12):

(SEQ ID NO:13)

TINVHDVETQFNQYKTEAASPYNLTISDVSVSDVPFPFSAQSGA

Truncated PSMGFR receptor (TR) (having “SPY” sequence of var-PSMGFR):

| | |
|---|---|
| GTINVHDVETQFNQYKTEAASPYNLTISDVSVS | (SEQ ID NO:14) |

Extended Sequence of MUC1 Growth Factor Receptor (ESMGFR) (having “SPY” sequence of var-PSMGFR):

(SEQ ID NO:15)

VQLTLAFREGTINVHDVETQFNQYKTEAASPYNLTISDVSVSDVPFPF

Tumor-Specific Extended Sequence of MUC1 Growth Factor Receptor (TSESMGFR) (having “SPY” sequence of var-PSMGFR):

(SEQ ID NO:16)

SVVVQLTLAFREGTINVHDVETQFNQYKTEAASPYNLTISDVSVSDVPFP
FSAQSGA

Primary Sequence of the Interchain Binding Region) (PSIBR):

| | |
|---|---|
| GFLGLSNIKFRPGSVVVQLTLAFRE | (SEQ ID NO:17) |

Truncated Interchain Binding Region) (TPSIBR):

| | |
|---|---|
| SVVVQLTLAFREG | (SEQ ID NO:18) |

Repeat Motif 2 (RM2):

(SEQ ID NO:19)

PDTRPAPGSTAPPAHGVTSAPDTRPAPGSTAPPAHGVTSA

What is claimed is:

1. A method of determining likelihood of acquiring drug resistance of a tumor or cancerous cells, cancer metastasis, cancer recurrence, or decreased life expectancy, comprising measuring the level of MUC1 or MUC1-associated factor expressed in the cancerous cells or tumor.

2. The method according to claim 1, comprising measuring an increase in the amount of MUC1 or MUC 1-associated factor that the cancerous cells or tumor produces.

3. The method according to claim 2, comprising using fluorescent in situ hybridization (FISH) technique to measure the ratio of MUC1 expression relative to CEP17 expression, wherein a ratio of greater than 1.8 indicates likelihood of acquiring drug resistance of a tumor or cancerous cells, cancer metastasis, cancer recurrence, or decreased life expectancy.

4. The method according to claim 3, wherein the ratio is 2.0 or greater.

5. The method according to claim 2 comprising using immunohistochemistry, FISH or a sandwich assay technique to measure an amount of MUC1 or MUC1-associated factor wherein increased risk is associated with a measurement of MUC1 or MUC1-associated factor that is greater than 2 standard deviations above similar measurements on cells or tissues from a normal population indicates likelihood of acquiring drug resistance of a tumor or cancerous cells, cancer metastasis, cancer recurrence, or decreased life expectancy.

6. The method according to claim 5, wherein when immunohistochemistry is used to measure the amount of MUC1 or MUC 1-associated factor, membrane staining shows moderate (++) to strong (+++) readings in greater than 10% of a tumor specimen.

7. The method according to claim 1, wherein the MUC1-associated factor is MUC1*, NM23, MMP-14 or ADAM-17 (TACE).

8. The method according to claim 6, wherein the MUC1-associated factor is MUC1.

9. The method according to claim 1, wherein the tumor or cancerous cells is biliary tract cancer; bladder cancer; brain cancer; breast cancer; cervical cancer; choriocarcinoma; colon cancer; endometrial cancer; esophageal cancer; gastric cancer; hematological neoplasms; multiple myeloma; AIDS-associated leukemias and adult T-cell leukemia lymphoma; intraepithelial neoplasms; liver cancer; lung cancer; lymphomas; neuroblastomas; oral cancer; ovarian cancer; pancreatic cancer; prostate cancer; rectal cancer; sarcomas; skin cancer; testicular cancer; thyroid cancer; or renal cancer.

10. The method according to claim 9, wherein the tumor or cancerous cells is breast, prostate, lung, ovarian, colorectal, renal, or brain cancer.

11. The method according to claim 10, wherein the tumor or cancerous cells is breast cancer.

12. A method of determining a cancer patient's suitability for treatment with a MUC1-targeting therapy, comprising measuring an amount of MUC1 or MUC1-associated factor that a patient's cancer cells or tumor expresses, wherein the MUC1 or MUC1-associated factor amount of ++ to +++ in more than 10% of the cancer cells sampled indicates that MUC1-targeting therapy is appropriate.

13. The method according to claim 12, comprising measuring an increase in the amount of MUC1 or MUC1-associated factor that the cancerous cells or tumor produces.

14. The method according to claim 13, wherein the MUC1-associated factor is MUC1*, NM23, MMP-14 or ADAM-17 (TACE).

15. The method according to claim 14, wherein the MUC1-associated factor is MUC1*.

16. The method according to claim 12, wherein the cancer is biliary tract cancer; bladder cancer; brain cancer; breast cancer; cervical cancer; choriocarcinoma; colon cancer; endometrial cancer; esophageal cancer; gastric cancer; hematological neoplasms; multiple myeloma; AIDS-associated leukemias and adult T-cell leukemia lymphoma; intraepithelial neoplasms; liver cancer; lung cancer; lymphomas; neuroblastomas; oral cancer; ovarian cancer; pancreatic cancer; prostate cancer; rectal cancer; sarcomas; skin cancer; testicular cancer; thyroid cancer; or renal cancer.

17. The method according to claim 16, wherein the cancer is breast cancer.

18. The method according to claim 17, further comprising measuring an amount of HER2 in the cancerous cells or tumor, wherein
immunohistochemistry staining for HER2 that is weak to moderate membrane staining in more than 10% of the tumor; or
FISH ratio of HER2 expression relative to CEP17 expression, wherein a ratio of 2 or greater HER2:CEP17, indicates that treatment with a HER2-targeting agent and a MUC1-targeting agent is appropriate.

19. The method according to claim 18, wherein
immunohistochemistry staining for HER2 that is moderate to strong membrane staining in more than 30% of the tumor; or
FISH ratio of HER2 expression relative to CEP17 expression, wherein a ratio of 3.0 or greater, HER2:CEP17, indicates that treatment with a HER2-targeting agent and a MUC1*-targeting agent is appropriate.

20. The method according to claim 12, wherein the treatment using the MUC1-targeting therapy further comprises treatment with Cisplatin, AraC, Etoposide, cyclophosphamide, taxol, or doxirubicin.

21. The method according to claim 20, comprising treatment with trastuzumab, Cisplatin, AraC, Etoposide, cyclophosphamide, taxol, or doxirubicin consisting of measuring an amount of MUC1* and an amount of HER2.

22. A method for reducing breast cancer tumor size which cells present both MUC1* and HER2, comprising contacting the tumor with an effective amount of a HER2 disabling agent and a MUC1* disabling agent.

23. The method according to claim 22, wherein the HER2 disabling agent is trastuzumab and the MUC1* disabling agent is a monovalent anti-MUC1 antibody.


SEQUENCE LISTING (19 sequences)

SEQ ID NO: 1 - protein, 1255 aa, Artificial Sequence
  Full-length MUC1 Receptor
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 19 aa, Artificial Sequence
  N-terminal MUC-1 signaling sequence
MTPGTQSPFFLLLLLTVLT

SEQ ID NO: 3 - protein, 23 aa, Artificial Sequence
  N-terminal MUC-1 signaling sequence
MTPGTQSPFFLLLLLTVLTVVTA

SEQ ID NO: 4 - protein, 23 aa, Artificial Sequence
  N-terminal MUC-1 signaling sequence
MTPGTQSPFFLLLLLTVLTVVTG

SEQ ID NO: 5 - protein, 146 aa, Artificial Sequence
  A truncated MUC1 receptor
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 6 - protein, 171 aa, Artificial Sequence
  A truncated MUC1 receptor
GFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 7 - protein, 275 aa, Artificial Sequence
  A truncated MUC1 receptor
ATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 8 - protein, 233 aa, Artificial Sequence
  A truncated MUC1 receptor
GSGHASSTPGGEKETSATQRSSVPSSTEKNAFNSSLEDPSTDYYQELQRDISEMFLQIYKQGGFLGLSNIKFRPGSVVVQLTLAFREGTINVHDMETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 9 - protein, 863 aa, Artificial Sequence
  A truncated MUC1 receptor
LDPRVRTSAPDTRPAPGSTAPQAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 10 - protein, 45 aa, Artificial Sequence
  Native Primary Sequence
GTINVHDVETQFNQYKTEAASRYNLTISDVSVSDVPFPFSAQSGA

SEQ ID NO: 11 - protein, 44 aa, Artificial Sequence
  Native Primary Sequence
TINVHDVETQFNQYKTEAASRYNLTISDVSVSDVPFPFSAQSGA

SEQ ID NO: 12 - protein, 45 aa, Artificial Sequence
  “SPY” functional variant
GTINVHDVETQFNQYKTEAASPYNLTISDVSVSDVPFPFSAQSGA

SEQ ID NO: 13 - protein, 44 aa, Artificial Sequence
  “SPY” functional variant
TINVHDVETQFNQYKTEAASPYNLTISDVSVSDVPFPFSAQSGA

SEQ ID NO: 14 - protein, 33 aa, Artificial Sequence
  Truncated PSMGFR receptor
GTINVHDVETQFNQYKTEAASPYNLTISDVSVS

SEQ ID NO: 15 - protein, 48 aa, Artificial Sequence
  Extended Sequence
VQLTLAFREGTINVHDVETQFNQYKTEAASPYNLTISDVSVSDVPFPF

SEQ ID NO: 16 - protein, 57 aa, Artificial Sequence
  Tumor-Specific Extended Sequence
SVVVQLTLAFREGTINVHDVETQFNQYKTEAASPYNLTISDVSVSDVPFPFSAQSGA

SEQ ID NO: 17 - protein, 25 aa, Artificial Sequence
  Primary Sequence
GFLGLSNIKFRPGSVVVQLTLAFRE

SEQ ID NO: 18 - protein, 13 aa, Artificial Sequence
  Truncated Interchain
SVVVQLTLAFREG

SEQ ID NO: 19 - protein, 40 aa, Artificial Sequence
  Repeat Motif 2
PDTRPAPGSTAPPAHGVTSAPDTRPAPGSTAPPAHGVTSA